(19) United States
(12) Patent Application Publication
(10) Pub. No.: US 2011/0045065 A1
(43) Pub. Date: Feb. 24, 2011

(54) SUBSTANCE HAVING ANTIOXIDANT, GEROPROTECTIVE AND ANTI-ISCHEMIC ACTIVITY AND METHOD FOR THE PREPARATION THEREOF

(76) Inventors: Ashok Vasantray Vyas, Vadodara (IN); Ravindra Tukaram Jahdav, Panvel (IN); Subhash Trimbak Phad, Hyderabad (IN); Michail Arkadievich Ostrovskiy, Moscow (RU)

Correspondence Address: LUCAS & MERCANTI, LLP, 475 PARK AVENUE SOUTH, 15TH FLOOR, NEW YORK, NY, 10016, US

(21) Appl. No.: 12/884,541

(22) Filed: Sep. 17, 2010

Related U.S. Application Data
(63) Continuation-in-part of application No. 12/338,342, filed on Dec. 18, 2008.
Application No. 12/338,342 is a continuation of application No. 11/995,285, now abandoned.

(51) Int. Cl. A61K 9/48; A61K 31/4412; A61K 9/00; A61K 9/28; A61K 9/14; A61K 9/16

(52) U.S. Cl. 424/452; 514/345; 424/43; 424/400; 424/465; 424/490

(57) ABSTRACT

The present invention relates to medicinal formulations having anti-oxidant, geroprotective and anti-ischemic activity. Said formulations include 3-hydroxy 2,4,6 trimethylpyridine, pharmaceutically acceptable salts, esters, derivatives and polymorphs thereof.

### RELATED U.S. APPLICATION DATA

This application is a continuation-in-part of U.S. application Ser. No. 12/338,342, filed on Dec. 18, 2008, which is a continuation of U.S. application Ser. No. 11/995,285, filed on Jan. 10, 2008, now abandoned, which is a U.S. national stage of PCT/IB2005/00363, filed on Nov. 7, 2005, the contents of which are incorporated herein by reference.

### FIELD OF THE INVENTION

The present invention relates to medicinal formulations having anti-oxidant, geroprotective and anti-ischemic activity.

### BACKGROUND OF THE INVENTION

Ischemia is restriction in blood supply due to factors in the blood vessels, with resultant damage or dysfunction of tissue. Ischemia is absolute or relative shortage of the blood supply to an organ. Relative shortage means mismatch between blood supply (oxygen delivery) and blood requirement for adequate oxygenation of tissue. Ischemia results in tissue damage because of lack of oxygen and nutrients. Ultimately, this causes great damage because of a buildup of metabolic wastes.

Ischemia can also be described as an inadequate flow of blood to a part of the body, caused by constriction or blockage of the blood vessels supplying it.

Depending on the type of organ that is affected, ischemia can be broadly classified in to the following categories:

1. Cardiac ischemia: Ischemia of heart muscle produces angina pectoris.

2. Bowel ischemia: An ischemia in the large bowel caused by an inflammation results in ischemic colitis and ischemia in the small bowel, caused by an inflammation results in mesenteric ischemia.

3. Cutaneous ischemia: Reduced blood flow to the skin layers may result in mottling or uneven, patchy discoloration of the skin.

4. Cerebral ischemia: is the localized reduction of blood flow to the brain or parts of the brain due to arterial obstruction or systematic hyperfusion.

Aging is a universal and inevitable phenomenon that affects all human beings. It can be considered as a product of an interaction between genetic, environmental and lifestyle factors, which in turn influence longevity. Sometimes result of the failure of homeostasis due to the accumulation of damage may lead to aging process.

Very few geroprotective compounds for slowing aging, prolonging lifespan of an individual or cells in an individual, and/or improving quality of life of an individual are known such as melatonin, N-acetylserotonin (NAS), pineal gland peptides [epithalamin and epitalon] and L-Ala-L-Glu-L-Asp-Gly (SEQ ID NO: 1) tetrapeptide.

Intraocular hemorrhage (hemophthalmos or hemophthalmia) is a condition in which bleeding occurs in the eyeball. It may be the result of physical trauma (direct injury to the eye) and/or medical illness. Severe hemorrhage, particularly when leading to rising pressure inside the eye, may lead to blindness. There are different types of intraocular hemorrhage such as subconjunctival hemorrhage, hyphema, vitreous hemorrhage, subretinal hemorrhage and submacular hemorrhage. Different causes responsible for bleeding in different locations are terson's syndrome (as a result of subarachnoid hemorrhage), hemophilia (a severe bleeding disorder, usually hereditary), anticoagulants and thrombolysis (medication to reduce blood clotting tendency or to disperse blood clots, respectively).

It has been proved that hemophthalmia is always accompanied by activation of free radical oxidation processes and proceeded as chain reactions and involves accumulation of oxidation products of molecules in vitreous body and retina. Application of antioxidant preparations in early conservative therapy of intraocular hemorrhages essentially allows to speed up the resorption processes and thus reduces the risk of development of serious complications such as fibrosis of vitreous body and retinal detachment.

In the last decade researches of have established that inflammatory reaction is always accompanied by activation of processes of free radical oxidation, mutually aggravating each other and resulting in development of various post-inflammatory complications. Use of antioxidant in early conservative therapy of inflammatory pathology of eye allows to speed up recovery and to improve the disease prognosis.

A medicine for antioxidant protection of media and tissues of the eye being a biosolvable polymer with a hydrophylic plasticizing additive, and comprising emoxypine and piroxydine hydrochloride as a drug is known (RU 2070010, 1993).

The bioactive food additive “glutapiron” having inter alia geroprotective activity, and comprising salts of 2-(2,6-dimethyl-3,5-dietoxycarbonile-1,4-dihydropyridine-4-carboxamido) pentane diacid as active agent is known (RU 95116403, 1995).

The use of dipotassic salt of N-(3-chloro-1,4-naphtohynonyl)-2-glutamic acid as an agent showing inter alia anti-ischemic activity is also known.

Closest prior art with respect to the present invention represents 2-methyl-6-ethyl-3-hydroxypyridine succinate (mexydole) (M. D. Mashkovsky, “Pharmaceuticals”, part 11, 1993, Moscow, Medicine, page 216), having antioxidant and membranoprotective action, said substance being prepared by heating of an alcoholic solution of 2-methyl-6-ethyl-3-hydroxypyridine with succinic acid for 1 hour (SU 509047, 1973).

### OBJECTS OF THE INVENTION

It is an object of the present invention to provide new substances being derivatives of 3-hydroxypyridine family having antioxidant, geroprotective and anti-ischemic properties.

It is another object of the present invention to provide a method for preparing derivatives of 3-hydroxypyridine family having antioxidant, geroprotective and anti-ischemic activities.

It is still another object of the present invention to provide geroprotective, anti-ischemic and antioxidant formulations comprising derivatives of 3-hydroxypyridine family.

It is yet another objective of the present invention to provide medicinal formulations for therapeutic and/or prophylactic purpose in various eye diseases.

It is another object of the present invention to provide medicinal formulations for the treatment of intraocular hemorrhage and macular degeneration.

It a further objective of the present invention to provide stable medicinal formulations capable of being delivered by various routes such as oral, topical, parenteral, intra-ocular and the like.

It is still further object of the present invention to provide medicinal formulations which do not cause irritation at the site of application.

### SUMMARY OF THE INVENTION

In accordance with the first aspect of the present invention, there is provided a pharmaceutically acceptable salt of 2,4,6-trimethyl-3-hydroxypyridine with a lower dicarboxylic acid, of formula (I).

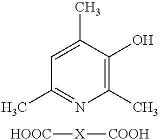

Wherein X is selected from the group consisting of a single bond, —CH₂—, —CH₂CH₂— and —CH₂CH(OH)—
having antioxidant, geroprotective and anti-ischemic activity.

Typically, the salt is selected from the group consisting of 2,4,6-trimethyl-3-oxypyridine oxalate, 2,4,6-trimethyl-3-oxypyridine malonate, 2,4,6-trimethyl-3-oxypyridine succinate and 2,4,6-trimethyl-3-oxypyridine malate.

In accordance with another aspect of the present invention, there is provided a method for preparing the 2,4,6-trimethyl-3-hydroxypyridine salt; said method comprising the following steps:
a. reacting equimolar amounts of 2,4,6-trimethyl-3-hydroxypyridine and a dicarboxylic acid in a solution of a lower alcohol at boiling temperature;
b. treating the resulting reaction mixture with an organic solvent; and
c. optionally, maintaining the reaction mixture for 2 to 5 hours at 10 to 15° C.

In accordance with still another aspect of the present invention, there is provided a medicinal formulation comprising a bioactive selected from the group consisting of 3-hydroxy-2,4,6 trimethylpyridine and pharmaceutically acceptable salts, esters, derivatives and polymorphs of 3-hydroxy-2,4,6 trimethylpyridine in an amount of about 0.05 to about 50% of the mass of the formulation and at least one pharmaceutically acceptable excipient selected from the group consisting of diluents, disintegrants, lubricants, glidents, binders, surfactants, solvents, coating polymers, effervescent agents, sweeteners, flavoring agents, colorants, preservatives stabilizer, thickening agents, chelating agents, buffering agents, tonicity agents, pH adjusting agents, ointment bases, oils, waxes and vehicle.

Typically, the pharmaceutically acceptable salt of 3-hydroxy-2,4,6-trimethylpyridine is selected from the group of salts consisting of succinate, maleate, tartrate, oxalate, fumarate, citrate, hydrochloride, salicylate, pamoate, hydrogen sulfate, sulfate methanesulphonate and benzenesulfonate.

Typically, the formulation is in a dosage form selected from the group of dosage forms consisting of tablet, oral liquid, capsule, powder, dry syrup, granules, ointment, gel, cream, ophthalmic preparation, injection, spray, emulsion and suspension.

In accordance with an embodiment of the present invention the formulation is in the form of a tablet and the pharmaceutically acceptable excipients therein comprises diluents, disintegrants, lubricants, glidents, binders, surfactants, solvents, matrix forming agents, coating polymers, effervescent agents, sweeteners, flavoring agents, colorants and preservatives.

Typically, the diluent is at least one selected from the group consisting of microcrystalline cellulose, starches, lactose, mannitol, calcium phosphate, dibasic calcium phosphate and mixture thereof.

Typically, the disintegrant is at least one selected from the group consisting of starches, clays, cellulose derivatives, gums, aligns including alginic acid, combinations of hydrocarbonates with weak acids, crospovidone, sodium starch glycolate, agar, cation exchange resins, citrus pulp, veegum HV, natural sponge, and bentonite cross-linked polyvinylpyrrolidone, carboxymethyl starch, natural starch, microcrystalline cellulose, cellulose gum, carboxymethylcellulose calcium, carboxymethylcellulose sodium, colloidal silica, colloidal silicon dioxide, croscarmellose sodium, guar gum, polacrilin potassium, pregelatinized starch, sodium alginate and sodium starch glycolate.

Typically, the lubricant is at least one selected from the group consisting of magnesium stearate, calcium stearate, glyceryl monostearate, glyceryl palmitostearate, stearic acid, talc, and zinc stearate, stearic acid, magnesium lauryl sulfate, and colloidal silicon dioxide.

Typically, the binder is at least selected from the group consisting of acacia, sodium alginate, starch, gelatin, pregelatinized starch, partly pregelatinized starch, saccharides, glucose, sucrose, dextrose, lactose, molasses, panwar gum, guar gum, ghatti gum, carboxy methylcellulose, methylcellulose, veegur, polyethylene glycols, ethylcellulose, polyvinylpyrrolidone, hydroxypropyl cellulose, hydroxypropyl methylcellulose, starch, gum arabic and dextrin.

Typically, the surfactant is at least one selected from the group consisting of alkyl polyethylene oxide, alkylphenol polyethylene oxide, sodium laureth sulphate, sodium dodecyl sulphate, alkyl alcohol, sodium lauryl sulfate, polyoxyethylene/polyoxypropylene block polymers (poloxamers), glycerols, polyglycerols, fatty acids, polyethylene glycol hydroxystearate, polyalkyl glucosides, ceramides, polyethylene glycol/alkyl glycol copolymers, and polyethylene glycol/polyalkylene glycol ether di-block or tri-block copolymers, diacetylated monoglycerides, diethylene glycol monostearate, ethylene glycol monostearate, glyceryl monooleate, propylene glycol monostearate, macrogol esters, macrogol stearate, polyoxyethylene 50 stearate, macrogol ethers, cetomacrogol 1000, lauromacrogols, nonoxinols, octoxinols, tyloxapol, polyvinyl alcohols, polysorbate, sorbitan monolaurate, sorbitan monooleate, sorbitan monopalmitate, sorbitan sesquioleate, sorbitan trioleate, sorbitan tristearate, sucrose esters, cetyl alcohol, oleyl alcohol, cetylpyridinium chloride, cetyl trimethylammonium bromide, tween 20 and tween 80.

Typically, the glidant is at least one selected from the group consisting of colloidal silicon dioxide, magnesium trisilicate, powdered cellulose, starch, talc, tribasic calcium phosphate, lactose, stearates, dibasic calcium phosphate, magnesium carbonate, magnesium oxide, calcium silicate and silicon dioxide aerogels.

Typically, the matrix forming agent (thickening agent) is at least one selected from the group consisting of hydroxypropyl methylcellulose, hydroxypropyl cellulose, sodium carboxy methyl cellulose, hydroxyethyl cellulose, polyethylene glycol, acrylates, methacrylates, gelatin, alginates, pectins, tragacanth, karaya gum, xanthan gum, carrageenin, agar, alginate, chitosan, eudrajit and acacia.

Typically, the coating polymer is at least one selected from the group consisting of polymethacrylate, polymethamethacrylate, ethyl cellulose, hydroxymethyl cellulose, hydroxymethylpropylcellulose, cellulose acetate phthalate, arabinogalactan, carboxymethylcellulose, gelatin, gum arabic, methylcellulose; polyvinyl alcohol, polyamide, silicones, polyvinyl acetate, hydroxypropyl methylcellulose acetate, rosin, partially hydrogenated rosin and glycerol esters of rosin.

Typically, the plasticizer is at least one selected from the group consisting of glycerol, polyethylene glycol, propylene glycol, sugar solution, alcohol, sorbitol, diethyl butyl pthalate, silicone, hexanol, pentanol, dimethylsulfoxide, hexane, oil and mixtures thereof.

Typically, the effervescent agent is at least one selected from the group consisting of citric acid, tartaric acid, sodium bicarbonate, potassium bicarbonate and calcium carbonate.

Typically, the sweetener is at least one selected from the group consisting of sodium saccharin, calcium saccharin, cyclamic acid, cyclamate salts, dihydrochalcones, L-aspartyl-L-phenylalanine methyl ester, glycyrrhizin, glycyrrhizic acid, ammonium salt, sorbitol, mannitol, xylitol, aspartame, potassium acesulfame, sodium saccharinate, neohesperidin dihydrochalcone and mixtures thereof.

Typically, the flavoring agent is at least one selected from the group consisting of anise oil, peppermint, lemon, mint, strawberry, banana, pineapple, orange, raspberry and vanilla.

In accordance with another embodiment of the present invention the formulation is in the form of a oral liquid and the pharmaceutically acceptable excipients therein comprises sucrose, sorbitol, glycerol, mannitol, propylene glycol, polyethylene glycol, saccharin, aspartame, methyl Paraben, propyl Paraben, sodium benzoate, sorbic acid, potassium sorbate, benzoic acid, carboxymethylcellulose sodium, hydroxypropylmethylcellulose, colloidal silica, methyl cellulose, gum tragacanth, gum acacia, sodium alginate, povidone, xanthan gum, guar gum, tween 80, simethicon, disodium edentate, citric acid, sodium citrate, sodium acetate, sodium phosphate, sodium hydroxide, hydrochloric acid, colorants and flavoring agents.

In accordance with still another embodiment of the present invention the formulation is in the form of a capsule and the pharmaceutically acceptable excipients therein comprises microcrystalline cellulose, lactose, starch, dicalcium phosphate, colloidal silica, magnesium stearate, talc, sodium startch Glycollate, Crospovidone, croscarmellose sodium, methyl paraben, propyl paraben, povidone and pregelatinised starch.

In accordance with a further embodiment of the present invention the formulation is in the form of a dry syrup and the pharmaceutically acceptable excipients therein comprises sucrose, mannitol, sorbitol solid, saccharin, aspartame, sodium benzoate, sorbic acid, potassium sorbate, carboxymethylcellulose sodium, hydroxypropylmethylcellulose, colloidal silica, methyl cellulose, gum tragacanth, gum acacia, sodium alginate, Povidone, xanthan gum, guar gum, disodium edentate, sodium lauryl sulphate, citric acid, sodium citrate, sodium acetate, colorants and flavoring agents.

In accordance with a still further embodiment of the present invention the formulation is in the form of a powder and the pharmaceutically acceptable excipients therein comprises lactose, microcrystalline cellulose, sucrose, mannitol, sorbitol solid, saccharin, aspartame, sodium benzoate, sorbic acid, potassium sorbate, citric acid, sodium citrate, sodium acetate, sodium bicarbonate, povidone, starch, colorants and flavoring agents.

In accordance with yet another embodiment of the present invention the formulation is in the form of ophthalmic preparation and the pharmaceutically acceptable excipients therein comprises preservative, stabilizer, thickening agents, chelating agents, buffering agent, surfactants, tonicity agents, pH adjusting agents and a vehicle.

In accordance with a preferred embodiment of the present invention the ophthalmic preparation comprises 0.05 to 10% of 3-hydroxy-2,4,6-trimethylpyridine, 0.005 to 0.5% of preservatives and 0.01 to 1% of stabilizer.

Typically, the pH of the ophthalmic preparation is in range of about 2.5 to about 7.5.

Typically, the preservative is at least one selected from the group consisting of benzalkonium chloride, benzyl alcohol, methyl paraben, propyl paraben, butyl paraben, chlorobutanol, metacresol, phenylmercuric nitrate, phenylmercuric acetate orphenylmercuric borate, thiomersal, myristylgamma picolonium chloride, phenol, benzoxonium chloride, cetrimide, phenyl ethanol, chlorohexidine, sorbic acid, potassium sorbate and sodium perborate.

Typically, the stabilizer is at least one selected from the group consisting of thiourea, thiosorbitol, sodium dioctyl sulfosuccinate or monothioglycerol, sodium edetate, creatinine, glycine, niacinamide, sodium acetyltryptophanate, sodium caprylate, sodium saccharin, citric acid and salts thereof.

Typically, the thickening agent is at least one selected from the group consisting of hydroxypropyl methylcellulose, hydroxypropyl cellulose, sodium carboxy methyl cellulose, hydroxyethyl cellulose, polyethylene glycol, acrylates, methacrylates, gelatin, alginates, pectins, tragacanth, karaya gum, xanthan gum, carrageenin, agar, alginate, chitosan and acacia.

Typically, the chelating agent is at least one selected from the group consisting of edetate disodium, edetate calcium disodium and edetate tetrasodium.

Typically, the buffering agent is at least one selected from the group consisting of acetic acid, adipic acid, benzoic acid, sodium benzoate, citric acid, lactic acid, maleic acid, potassium phosphate, sodium phosphate, sodium acetate, sodium bicarbonate, sodium carbonate, sodium citrate, sodium tartarate, tartaric acid, sodium citrate dehydrate, sodium acetate, and sodium acetate trihydrate.

Typically, the surfactant is at least one selected from the group consisting of alkyl polyethylene oxide, alkylphenol polyethylene oxide, sodium laureth sulphate, sodium dodecyl sulphate, alkyl alcohol, sodium lauryl sulfate, polyoxyethylene/polyoxypropylene block polymers (poloxamers), glycerols, polyglycerols, fatty acids, polyethylene glycol hydroxystearate, polyalkyl glucosides, ceramides, polyethylene glycol/alkyl glycol copolymers, and polyethylene glycol/polyalkylene glycol ether di-block or tri-block copolymers, diacetylated monoglycerides, diethylene glycol monostearate, ethylene glycol monostearate, glyceryl monooleate, propylene glycol monostearate, macrogol esters, macrogol stearate, polyoxyethylene 50 stearate, macrogol ethers, cetomacrogol 1000, lauromacrogols, nonoxinols, octoxinols, tyloxapol, polyvinyl alcohols, polysorbate, sorbitan monolaurate, sorbitan monooleate, sorbitan monopalmitate, sorbitan sesquioleate, sorbitan trioleate, sorbitan tristearate, sucrose esters, cetyl alcohol, oleyl alcohol, cetylpyridinium chloride, cetyl trimethylammonium bromide, tween 20 and tween 80.

Typically, the tonicity agent is at least one selected from the group consisting of glycerin, propylene glycol, polyethylene glycol, lactose, mannitol, dextrose, sodium chloride, potassium chloride, calcium chloride, magnesium chloride, sodium sulfate and sorbitol.

Typically, the pH adjusting agent is selected from the group consisting of sodium hydroxide, hydrochloric acid, triethanolamine, ammonia and mixtures thereof.

In accordance with one of the embodiments of the present invention the vehicle is water for injection and said composition is in the form of a solution.

In accordance with another embodiment of the present invention the vehicle is at least one ointment base selected from the group consisting of oil and wax and the ophthalmic preparation is in the form of an ointment.

Typically, the oil is at least one selected from the group consisting of isopropyl myristate, myristyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, ethylhexyl cocoate, dicaprylyl carbonate, cetearyl isononanoate, oleyl erucate, erucyl oleate, erucyl erucate, octyldodecanol, polydecenes, squalane, dicaprylyl ether, triisostearine, butylene glycol dicaprylate/dicaprate, caprylic/capric triglyceride, olive oil, sunflower oil, soybean oil, peanut oil, rapeseed oil, almond oil, palm oil, coconut oil, palm karnel oil, caster oil, wheat germ oil, grape seed oil, thistle oil, silicon oils, lanolin oil, avocado oil and macadamia oil.

Typically, the wax is at least one selected from the group of candelilla wax, carnauba wax, bees wax, espartograss wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresin, ozocerite (earth wax), paraffin waxes, sunflower wax, lemon wax, grape fruit wax and laurel wax.

In accordance with another embodiment of the present invention the vehicle comprises at least one polymer selected from the group consisting hydroxypropyl methylcellulose, hydroxypropyl cellulose, sodium carboxy methyl cellulose, hydroxyethyl cellulose, carbopol and the ophthalmic preparation is in the form of a gel.

In accordance with the still another embodiment of the present invention the formulation is in the form of injection and the pharmaceutically acceptable excipients therein comprises preservative, tonicity agent and water for injection.

In accordance with a preferred embodiment of the present invention the injection comprises 0.5 to 10% of 3-hydroxy-2,4,6-trimethylpyridine and 0.001 to 0.5% of preservatives.

Typically, the pH of the injection prepared in accordance with the present invention is in range of about 2.5 to about 7.5.

Typically, the preservative is at least one selected from the group consisting of benzalkonium chloride, benzyl alcohol, methyl paraben, propyl paraben, butyl paraben, chlorobutanol, metacresol, phenylmercuric nitrate, thiomersal, myristylgamma picolonium chloride, sorbic acid, potassium sorbate and phenol.

Typically, the tonicity agent is at least one selected from the group consisting of glycerin, lactose, mannitol, dextrose, sodium chloride, sodium sulfate and sorbitol.

### DETAILED DESCRIPTION OF THE INVENTION

The present invention provide new compounds representing pharmaceutically acceptable 2,4,6-trimethyl-3-hydroxypyridine salts with lower dicarboxylic acids of the general formula Ia-d having antioxidant, geroprotective and anti-ischemic activities:

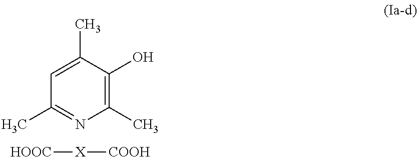

with X being a simple bond (compound Ia, oxalate, C₈H₁₁NO.C₂H₂O₄);

with X being CH₂ (compound Ib, malonate, C₈H₁₁NO.C₃H₄O₄);

with X being CH₂CH₂ (compound Ic, succinate, C₈H₁₁NO.C₄H₆O₄);

with X being the group CH₂CH(OH) (compound Id, malate, C₈H₁₁NO.C₄H₆O₅).

In accordance with another aspect of the present invention, there is provided a method for preparing the 2,4,6-trimethyl-3-hydroxypyridine salt; said method comprising the following steps:
a. reacting equimolar amounts of 2,4,6-trimethyl-3-hyroxypyridine and a dicarboxylic acid in a solution of a lower alcohol at boiling temperature;
b. treating the resulting reaction mixture with an organic solvent; and
c. optionally, maintaining the reaction mixture for 2 to 5 hours at 10 to 15° C.

Compounds Ia-d show antioxidant activity regarding the process of peroxide oxidation of lipids, that is appropriate to all 3-oxypyridine derivatives. This property allows to assume, that compounds Ia-d, as well as their analogs emoxypine (hydrochloride 2-methyl-6-ethyl-3-hydroxypyridine) and mexydole (2-methyl-6-ethyl-3-hydroxypyridine succinate), can be used as medical agents in opthalmology, namely, at subconjunctival and ocular hemorrhages into any parts and tissues of the eye, angioretinopathies of different etiologies, including diabetic, chorioretinal dystrophies, retinal central vein thrombosis and vein branches, complicated myopia, dystrophy keratites, and for protection and treatment of cornea and retina under the influence of light of high effectiveness after operations with retinal detachment. Furthermore, compound Ic having expressed anti-ischemic activity is assumed to be possibly used in cardiology for treating ischemic heart disease and atherosclerosis.

Compound Ic has also been found to be an active inhibitor of photoinduced peroxide oxidation of lipids sensitized by “senile pigment” being lipofuscin granules, isolated from eyes of elderly people indicating that compound Ic is capable of neutralizing toxic activity of lipofuscin granules accumulated in senior age, i.e. to have geroprotective action. Thus, it is possible to assume, that compound Ic may be used to inactivate the toxic action of lipofuscin. The lipofuscin, as now commonly accepted, is a basic factor leading to development of senile macular retinal degeneration. The senile macular retinal degeneration belongs to the most widespread ophthalmic diseases of people above the age of 60 years. According to statistical data said disease leading to full blindness, affects almost 30% of the Americans at the age of above 65 years, and the percentage of diseased people fast increases with the age.

In accordance with still another aspect of the present invention, there is provided a medicinal formulation comprising a bioactive selected from the group consisting of 3-hydroxy-2,4,6 trimethylpyridine and pharmaceutically acceptable salts, esters, derivatives and polymorphs of 3-hydroxy-2,4,6 trimethylpyridine in an amount of about 0.05 to about 50% of the mass of the formulation and at least one pharmaceutically acceptable excipient selected from the group consisting of diluents, disintegrants, lubricants, glidents, binders, surfactants, solvents, matrix forming agents, coating polymers, effervescent agents, sweeteners, flavoring agents, colorants, preservatives stabilizer, thickening agents, chelating agents, buffering agents, tonicity agents, pH adjusting agents, ointment bases, oils, waxes and vehicle.

Typically, the pharmaceutically acceptable salt of 3-hydroxy-2,4,6-trimethylpyridine is selected from the group of salts consisting of succinate, maleate, tartrate, oxalate, fumarate, citrate, hydrochloride, salicylate, pamoate, hydrogen sulfate, sulfate methanesulphonate and benzenesulfonate.

Typically, the formulation is in a dosage form selected from the group of dosage forms consisting of tablet, oral liquid, capsule, powder, dry syrup, granules, ointment, gel, cream, ophthalmic preparation, injection, spray, emulsion and suspension.

In accordance with an embodiment of the present invention the formulation is in the form of a tablet and the pharmaceutically acceptable excipients therein comprises diluents, disintegrants, lubricants, glidents, binders, surfactants, solvents, matrix forming agents, coating polymers, effervescent agents, sweeteners, flavoring agents, colorants and preservatives.

Typically, the diluent is at least one selected from the group consisting of microcrystalline cellulose, starches, lactose, mannitol, calcium phosphate, dibasic calcium phosphate and mixture thereof.

Typically, the disintegrant is at least one selected from the group consisting of starches, clays, cellulose derivatives, gums, aligns including alginic acid, combinations of hydrocarbonates with weak acids, crospovidone, sodium starch glycolate, agar, cation exchange resins, citrus pulp, veegum HV, natural sponge, and bentonite cross-linked polyvinylpyrrolidone, carboxymethyl starch, natural starch, microcrystalline cellulose, cellulose gum, carboxymethylcellulose calcium, carboxymethylcellulose sodium, colloidal silica, colloidal silicon dioxide, croscarmellose sodium, guar gum, polacrilin potassium, pregelatinized starch, sodium alginate and sodium starch glycolate.

Typically, the lubricant is at least one selected from the group consisting of magnesium stearate, calcium stearate, glyceryl monostearate, glyceryl palmitostearate, stearic acid, talc, and zinc stearate, stearic acid, magnesium lauryl sulfate, and colloidal silicon dioxide.

Typically, the binder is at least selected from the group consisting of acacia, sodium alginate, starch, gelatin, pregelatinized starch, partly pregelatinized starch, saccharides, glucose, sucrose, dextrose, lactose, molasses, panwar gum, guar gum, ghatti gum, carboxy methylcellulose, methylcellulose, veegur, polyethylene glycols, ethylcellulose, polyvinylpyrrolidone, hydroxypropyl cellulose, hydroxypropyl methylcellulose, starch, gum arabic and dextrin.

Typically, the surfactant is at least one selected from the group consisting of alkyl polyethylene oxide, alkylphenol polyethylene oxide, sodium laureth sulphate, sodium dodecyl sulphate, alkyl alcohol, sodium lauryl sulfate, polyoxyethylene/polyoxypropylene block polymers (poloxamers), glycerols, polyglycerols, fatty acids, polyethylene glycol hydroxystearate, polyalkyl glucosides, ceramides, polyethylene glycol/alkyl glycol copolymers, and polyethylene glycol/polyalkylene glycol ether di-block or tri-block copolymers, diacetylated monoglycerides, diethylene glycol monostearate, ethylene glycol monostearate, glyceryl monooleate, propylene glycol monostearate, macrogol esters, macrogol stearate, polyoxyethylene 50 stearate, macrogol ethers, cetomacrogol 1000, lauromacrogols, nonoxinols, octoxinols, tyloxapol, polyvinyl alcohols, polysorbate, sorbitan monolaurate, sorbitan monooleate, sorbitan monopalmitate, sorbitan sesquioleate, sorbitan trioleate, sorbitan tristearate, sucrose esters, cetyl alcohol, oleyl alcohol, cetylpyridinium chloride, cetyl trimethylammonium bromide, tween 20 and tween 80.

Typically, the glidant is at least one selected from the group consisting of colloidal silicon dioxide, magnesium trisilicate, powdered cellulose, starch, talc, tribasic calcium phosphate, lactose, stearates, dibasic calcium phosphate, magnesium carbonate, magnesium oxide, calcium silicate and silicon dioxide aerogels.

Typically, the matrix forming agent (thickening agent) is at least one selected from the group consisting of hydroxypropyl methylcellulose, hydroxypropyl cellulose, sodium carboxy methyl cellulose, hydroxyethyl cellulose, polyethylene glycol, acrylates, methacrylates, gelatin, alginates, pectins, tragacanth, karaya gum, xanthan gum, carrageenin, agar, alginate, chitosan, eudrajit and acacia.

Typically, the coating polymer is at least one selected from the group consisting of polymethacrylate, polymethamethacrylate, ethyl cellulose, hydroxymethyl cellulose, hydroxymethylpropylcellulose, cellulose acetate phthalate, arabinogalactan, carboxymethylcellulose, gelatin, gum arabic, methylcellulose; polyvinyl alcohol, polyamide, silicones, polyvinyl acetate, hydroxypropyl methylcellulose acetate, rosin, partially hydrogenated rosin and glycerol esters of rosin.

Typically, the plasticizer is at least one selected from the group consisting of glycerol, polyethylene glycol, propylene glycol, sugar solution, alcohol, sorbitol, diethyl butyl pthalate, silicone, hexanol, pentanol, dimethylsulfoxide, hexane, oil and mixtures thereof.

Typically, the effervescent agent is at least one selected from the group consisting of citric acid, tartaric acid, sodium bicarbonate, potassium bicarbonate and calcium carbonate.

Typically, the sweetener is at least one selected from the group consisting of sodium saccharin, calcium saccharin, cyclamic acid, cyclamate salts, dihydrochalcones, L-aspartyl-L-phenylalanine methyl ester, glycyrrhizin, glycyrrhizic acid, ammonium salt, sorbitol, mannitol, xylitol, aspartame, potassium acesulfame, sodium saccharinate, neohesperidin dihydrochalcone and mixtures thereof.

Typically, the flavoring agent is at least one selected from the group consisting of anise oil, peppermint, lemon, mint, strawberry, banana, pineapple, orange, raspberry and vanilla.

In accordance with another embodiment of the present invention the formulation is in the form of a oral liquid and the pharmaceutically acceptable excipients therein comprises sucrose, sorbitol, glycerol, mannitol, propylene glycol, polyethylene glycol, saccharin, aspartame, methyl Paraben, propyl Paraben, sodium benzoate, sorbic acid, potassium sorbate, benzoic acid, carboxymethylcellulose sodium, hydroxypropylmethylcellulose, colloidal silica, methyl cellulose, gum tragacanth, gum acacia, sodium alginate, povidone, xanthan gum, guar gum, tween 80, simethicon, disodium edentate, citric acid, sodium citrate, sodium acetate, sodium phosphate, sodium hydroxide, hydrochloric acid, colorants and flavoring agents.

In accordance with still another embodiment of the present invention the formulation is in the form of a capsule and the pharmaceutically acceptable excipients therein comprises microcrystalline cellulose, lactose, starch, dicalcium phosphate, colloidal silica, magnesium stearate, talc, sodium startch Glycollate, Crospovidone, croscarmellose sodium, methyl paraben, propyl paraben, povidone and pregelatinised starch.

In accordance with a further embodiment of the present invention the formulation is in the form of a dry syrup and the pharmaceutically acceptable excipients therein comprises sucrose, mannitol, sorbitol solid, saccharin, aspartame, sodium benzoate, sorbic acid, potassium sorbate, carboxymethylcellulose sodium, hydroxypropylmethylcellulose, colloidal silica, methyl cellulose, gum tragacanth, gum acacia, sodium alginate, Povidone, xanthan gum, guar gum, disodium edentate, sodium lauryl sulphate, citric acid, sodium citrate, sodium acetate, colorants and flavoring agents.

In accordance with a still further embodiment of the present invention the formulation is in the form of a powder and the pharmaceutically acceptable excipients therein comprises lactose, microcrystalline cellulose, sucrose, mannitol, sorbitol solid, saccharin, aspartame, sodium benzoate, sorbic acid, potassium sorbate, citric acid, sodium citrate, sodium acetate, sodium bicarbonate, povidone, starch, colorants and flavoring agents.

In accordance with yet another embodiment of the present invention the formulation is in the form of ophthalmic preparation and the pharmaceutically acceptable excipients therein comprises preservative, stabilizer, thickening agents, chelating agents, buffering agent, surfactants, tonicity agents, pH adjusting agents and a vehicle.

In accordance with a preferred embodiment of the present invention the ophthalmic preparation comprises 0.05 to 10% of 3-hydroxy-2,4,6-trimethylpyridine, 0.005 to 0.5% of preservatives and 0.01 to 1% of stabilizer.

Typically, the pH of the ophthalmic preparation is in range of about 2.5 to about 7.5.

Typically, the preservative is at least one selected from the group consisting of benzalkonium chloride, benzyl alcohol, methyl paraben, propyl paraben, butyl paraben, chlorobutanol, metacresol, phenylmercuric nitrate, phenylmercuric acetate orphenylmercuric borate, thiomersal, myristylgamma picolonium chloride, phenol, benzoxonium chloride, cetrimide, phenyl ethanol, chlorohexidine, sorbic acid, potassium sorbate and sodium perborate.

Typically, the stabilizer is at least one selected from the group consisting of thiourea, thiosorbitol, sodium dioctyl sulfosuccinate or monothioglycerol, sodium edetate, creatinine, glycine, niacinamide, sodium acetyltryptophanate, sodium caprylate, sodium saccharin, citric acid and salts thereof.

Typically, the thickening agent is at least one selected from the group consisting of hydroxypropyl methylcellulose, hydroxypropyl cellulose, sodium carboxy methyl cellulose, hydroxyethyl cellulose, polyethylene glycol, acrylates, methacrylates, gelatin, alginates, pectins, tragacanth, karaya gum, xanthan gum, carrageenin, agar, alginate, chitosan and acacia.

Typically, the chelating agent is at least one selected from the group consisting of edetate disodium, edetate calcium disodium and edetate tetrasodium.

Typically, the buffering agent is at least one selected from the group consisting of acetic acid, adipic acid, benzoic acid, sodium benzoate, citric acid, lactic acid, maleic acid, potassium phosphate, sodium phosphate, sodium acetate, sodium bicarbonate, sodium carbonate, sodium citrate, sodium tartarate, tartaric acid, sodium citrate dehydrate, sodium acetate and sodium acetate trihydrate.

Typically, the surfactant is at least one selected from the group consisting of alkyl polyethylene oxide, alkylphenol polyethylene oxide, sodium laureth sulphate, sodium dodecyl sulphate, alkyl alcohol, sodium lauryl sulfate, polyoxyethylene/polyoxypropylene block polymers (poloxamers), glycerols, polyglycerols, fatty acids, polyethylene glycol hydroxystearate, polyalkyl glucosides, ceramides, polyethylene glycol/alkyl glycol copolymers, and polyethylene glycol/polyalkylene glycol ether di-block or tri-block copolymers, diacetylated monoglycerides, diethylene glycol monostearate, ethylene glycol monostearate, glyceryl monooleate, propylene glycol monostearate, macrogol esters, macrogol stearate, polyoxyethylene 50 stearate, macrogol ethers, cetomacrogol 1000, lauromacrogols, nonoxinols, octoxinols, tyloxapol, polyvinyl alcohols, polysorbate, sorbitan monolaurate, sorbitan monooleate, sorbitan monopalmitate, sorbitan sesquioleate, sorbitan trioleate, sorbitan tristearate, sucrose esters, cetyl alcohol, oleyl alcohol, cetylpyridinium chloride, cetyl trimethylammonium bromide, tween 20 and tween 80.

Typically, the tonicity agent is at least one selected from the group consisting of glycerin, propylene glycol, polyethylene glycol, lactose, mannitol, dextrose, sodium chloride, potassium chloride, calcium chloride, magnesium chloride, sodium sulfate and sorbitol.

Typically, the pH adjusting agent is selected from the group consisting of sodium hydroxide, hydrochloric acid, triethanolamine, ammonia and mixtures thereof.

In accordance with one of the embodiments of the present invention the vehicle is water for injection and said composition is in the form of a solution.

In accordance with another embodiment of the present invention the vehicle is at least one ointment base selected from the group consisting of oil and wax and the ophthalmic preparation is in the form of an ointment.

Typically, the oil is at least one selected from the group consisting of isopropyl myristate, myristyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, ethylhexyl cocoate, dicaprylyl carbonate, cetearyl isononanoate, oleyl erucate, erucyl oleate, erucyl erucate, octyldodecanol, polydecenes, squalane, dicaprylyl ether, triisostearine, butylene glycol dicaprylate/dicaprate, caprylic/capric triglyceride, olive oil, sunflower oil, soybean oil, peanut oil, rapeseed oil, almond oil, palm oil, coconut oil, palm karnel oil, caster oil, wheat germ oil, grape seed oil, thistle oil, silicon oils, lanolin oil, avocado oil and macadamia oil.

Typically, the wax is at least one selected from the group of candelilla wax, carnauba wax, bees wax, espartograss wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresin, ozocerite (earth wax), paraffin waxes, sunflower wax, lemon wax, grape fruit wax and laurel wax.

In accordance with another embodiment of the present invention the vehicle comprises at least one polymer selected from the group consisting hydroxypropyl methylcellulose, hydroxypropyl cellulose, sodium carboxy methyl cellulose, hydroxyethyl cellulose, carbopol and the ophthalmic preparation is in the form of a gel.

In accordance with the still another embodiment of the present invention the formulation is in the form of injection and the pharmaceutically acceptable excipients therein comprises preservative, tonicity agent and water for injection.

In accordance with a preferred embodiment of the present invention the injection comprises 0.5 to 10% of 3-hydroxy-2,4,6-trimethylpyridine and 0.001 to 0.5% of preservatives.

Typically, the pH of the injection prepared in accordance with the present invention is in range of about 2.5 to about 7.5.

Typically, the preservative is at least one selected from the group consisting of benzalkonium chloride, benzyl alcohol, methyl paraben, propyl paraben, butyl paraben, chlorobutanol, metacresol, phenylmercuric nitrate, thiomersal, myristylgamma picolonium chloride and phenol.

Typically, the tonicity agent is at least one selected from the group consisting of glycerin, lactose, mannitol, dextrose, sodium chloride, sodium sulfate and sorbitol.

Following examples illustrate the invention, but are not intended to limit the scope of the present invention.

### Example 1

### Preparation of Organic Salts of 2,4,6-trimethyl-3-oxypyridine

### A) Preparation of 2,4,6-trimethyl-3-oxypyridine oxalate (Ia)

0.8 g (0.00583 mol) 2,4,6-trimethyl-3-oxypyridine base, 0.525 g (0.00583 mol) waterless oxalic acid and 10 ml of methanol are fed to a flask with a magnetic stirrer and reflux condenser. Under stirring the reaction mixture is heated to boiling and kept boiling for 0.5 hours. Subsequently, the heating is stopped, the solvent is stripped off in vacuum, and 5 ml of acetone are added to the reaction mixture, and the mixture is triturated with a glass rod for 5-10 min. The resulting crystalline residue is separated by filtrating and is washed on the filter with acetone, and is dried in vacuum.

1.1 g (83% of the theory) of the salt are obtained. The melting temperature is 140-142.degree. C. Obtained, %: C, 53.4; H, 5.9. Calculated, %: C, 52.9; H, 5.8.

NMR-¹H Bruker WM-400 (400 MHz) (DMSO-d₆): 2.20 (3H, s, CH₃); 2.34 (3H, s, CH₃); 2.37 (3H, s, CH₃); 7.05 ppm (1H, s, CH).

### B) Preparation of 2,4,6-trimethyl-3-oxypyridine malonate (Ib)

0.8 g (0.00583 mol) 2,4,6-trimethyl-3-oxypyridine base, 0.607 g (0.00583 mol) malonic acid and 10 ml ethanol are fed to a flask with a magnetic stirrer and reflux condenser. Under stirring the reaction mixture is heated to boiling and kept boiling for 0.5 hours. Subsequently, the heating is stopped, the solvent is stripped off in vacuum, and 5 ml of acetone are added to the reaction mixture, the mixture being maintained for 3 hours at 10-15° C. The resulting crystalline residue is triturated, separated by filtrating, and is washed on the filter with acetone and dried in vacuum. 1.2 g (85.3% from the theory) of the salt are obtained. The melting temperature is 118-120.degree. C. Obtained, %: C, 55.0; H, 6.5. Calculated, %: C, 54.8; H, 6.3.

NMR-¹H Bruker WM-400 (400 MHz) (DMSO-d₆): 2.16 (3H, s, CH₃); 2.30 (3H, s, CH₃); 2.33 (3H, s, CH₃); 3.11 (2H, s, CH₂); 6.93 ppm (1H, s, CH).

### C) Preparation of 2,4,6-trimethyl-3-oxypyridine succinate (Ic)

3.4 g (0.025 mol) of 2,4,6-trimethyl-3-oxypyridine base, 2.93 g of (0.025 mol) succinic acid and 50 ml of izopropanol are fed to a flask with a magnetic stirrer and reflux condenser. Under stirring the reaction mixture is heated to boiling and kept boiling for 0.5 hours. Subsequently, the heating is stopped and 30 ml of acetone are stepwise added to the reaction mass, the mass being maintained for 3 hours at 10-15° C. The resulting crystalline residue is separated by filtrating, and is washed on the filter with acetone, and dried in vacuum. 5.6 g (88.5% from the theory) of the salt are obtained. The melting temperature is 128-129° C. Obtained, %: C, 56.5; H, 6.9. Calculated, %: C, 56.5; H, 6.7.

NMR-¹H (DMSO-d₆): 2.12 (3H, s, CH₃), 2.26 (3H, s, CH.sub.3), 2.31 (3H, s, CH₃), 2.42 (4H, s, CH₂), 6.79 (1H, s, CH-Py), 10.57 (b, s, COOH).

### D) Preparation of 2,4,6-trimethyl-3-oxypyridine malate (Id)

0.8 g (0.00583 mol) of 2,4,6-trimethyl-3-oxypyridine base, 0.782 g (0.00583 mol) of hydroxy-butanedioic acid and 10 ml of methanol are fed to a flask with a magnetic stirrer and reflux condenser. Under stirring the reaction mixture is heated to boiling and kept boiling for 0.5 hours. Subsequently, the solvent is removed in vacuum. The oily residue is twice washed with acetone under stirring, and the solvent is removed by decantation. The resulting mass is vacuumed and maintained for one hour at residual pressure of 0.5 mm Hg. Subsequently, the mass is maintained for crystallization for 48-72 hours. A white solid compound with a melting temperature of 70-75° C. is obtained.

The yield is 1.12 g (70.8% from the theory). Obtained, %: C, 53.5; H, 6.4. Calculated, %: 53.1; H, 6.3.

NMR-¹H Bruker WM-400 (400 MHz) (DMSO-d₆): 2.12 (3H, s, CH₃); 2.26 (3H, s, CH₃); 2.30 (3H, s, CH₃); 3.11 (2H, s, CH₂); 2.41 (1H, doublet dubl. J²_{HAHB}=15.7 Hz; J³_{HACH}=5.4 Hz CH_{A}H_{B}); 4.2 ppm (1H, doublet dubl. J³_{CHHA}=5.4 Hz; J³_{CHHB}=7.4 Hz, CH).

### Example 2

### Geroprotective Action of Compound 1c by the Example of Inhibition of Photoinduced Human Lipofuscin Granules of Liposome Peroxidation

Liposomes are prepared from methanolic solution of cardiolipin (initial concentration: 5 mg/ml) by evaporation of methanol and solubilization of cardiolipin in a phosphate buffer. A mixture comprising a suspension of liposomes and lipofuscin granules, isolated from tissue of a retinal pigmentary epithelium of a human eye, is subjected to irradiation with intensive blue light under constant stirring. Concentration of lipid peroxidation products (TBK-active products) is determined after 20, 40, 60 and 90 minutes. The experimental sample contained 2 mM of a solution of compound Ic, or 2 mM of a solution of mexydole. The results of the experiment are listed in table 1.

TABLE 1
Inhibiting effect of compound Ic (in comparison to mexydole)
regarding lipofuscin induced cardiolipin photoperoxidation

| Concentration of TBK-active products, | | | | | |
|---|---|---|---|---|---|
| nmol/mg lipid | | | | Inhibition, % | |
| Radiation | | Compound | | Compound | |
| time, min | Control | Ic | Mexydole | Ic | Mexydole |
| 0 | 1.83 | 1.3 | 1.83 | 0 | 0 |
| 20 | 3.0 | 2.42 | 2.84 | 20 | 5 |
| 40 | 3.5 | 2.53 | 3.5 | 28 | 0 |
| 60 | 4.52 | 2.85 | 4.5 | 40 | 0 |
| 90 | 5.38 | 3.28 | 5.0 | 40 | 7 |

The results indicate a protective effect of compound I regarding phototoxic action of the senile pigment-lipofuscin granules of the human eye. Under these conditions Mexydole practically did not show an inhibiting effect.

### Example 3

### Antioxidant Action of Compound Ic with Respect to Ascorbate Induced Peroxidation of Photoreceptor Cells of a Pig's Eye

The external segments of photoreceptor cells are obtained from pig retinas according to standard procedure, using methods of differential centrifuging in a saccharose density gradient. A mixture comprising a sodium-phosphate buffer (pH 7.3), 10⁸ segments/ml of external segments of photoreceptor cells, 0.5 mM ascorbic acid and 17 mM of compound Ic, was incubated in darkness under constant stirring for 15 and 30 minutes. Subsequently, the process was stopped by 15% trichloroacetic acid, and the concentration of TBK-active products has been determined. Samples being free of compound Ic were used as a control. The obtained results are shown in table 2a.

TABLE 2a
Inhibiting effect of compound Ic with respect to ascorbate-induced
peroxidation of external segments of photoreceptor cells
Concentration of TBK-active products relative units

| Response | | Compound | |
|---|---|---|---|
| time, min | Control | IB | Inhibition, % |
| 0 | 1 | 1 | — |
| 15 | 4.37 | 2.70 | 49.6 |
| 30 | 7.04 | 5.09 | 32.3 |

The comparison of antioxidant activity of compounds Ia, Ib, Ic and Id is shown in table 2b. In these experiments a comparison of the speed of ascorbate-induced peroxidation of external segments of photoreceptor cells of pig eyes has been carried out, said comparison being determined with respect to the accumulation of TBK-active products in the presence of different compound I salts.

TABLE 2b
Comparison of an inhibiting effect of compounds Ia, b, c, d with respect to
ascorbate-induced peroxidation of external segments of photoreceptor cells

| | Speed of accumulation of TBK-, | |
|---|---|---|
| Compound I | active products for 30 min of | |
| concentration, mM | reacting, nmol/mg | Inhibition, % |
| Control, 0 mM | 0.77 | — |
| Compound Ia, 44 mM | 0 | 100 |
| Compound Ib, 44 mM | 0.23 | 70 |
| Compound Ic, 43 mM | 0.05 | 93.5 |
| Compound Id, 43 mM | 0.1 | 87 |

The results indicate that all compound I salts show pronounced antioxidant activity regarding dark peroxidation of photoreceptor eye cells.

### Example 4

### Determination of Antiradical Activity of Compound Ic in Comparison with Mexydole

The efficiency of 3-hydroxypyridine derivatives as inhibitors of free radical reactions was determined by hemiluminescent method by means of measurement of speed constants of their reaction with ethyl-benzene k₇ peroxide radicals.

The measurements of intensity of chemiluminescence (HL) have been carried out with an apparatus CNK-7, designed and made by IHF RAN USSR. A photomultiplier FAU-38 is used as an optical receiver. The fixed concentration of radicals during oxidation of isopropyl toluene (cumole) is controlled by an initiator being azo-bis-isobutyronitrile.

In order to increase HL emission the activator chelate Eu (europium tristenoyl triphtoroacetonate with 1,10-phenatroline) has been used that has allowed to conduct measurements at low speed values of radical initiation (W₁=10⁻⁸-10⁻⁹ mol/I.s.) and, therefore, at small amounts of added sample. A weighed amount of samples studied was diluted in an suitable solvent (chlorobenzene or acetonitrile) and a small amount of the prepared solution (0.1-0.25 ml) was added to the reaction mixture (5-6 ml), arranged in a temperature-controlled reaction vessel of the HL-device. The change of emission intensity was recorded. The obtained results are shown in table 3.

TABLE 3
Antiradical activity of p-oxyderivatives of nitrogen heterocycles
with respect to ethyl-benzene peroxide radicals

| Compound | k₇ × 10⁻⁴ l/mol × sec |
|---|---|
| Metoxydole | 4.5 |
| Compound Ic | 9.5 |

The comparative evaluation of antiradical activity of compound I and mexydole has shown, that compound I shows a two times higher antiradical activity.

### Example 5

### Study of the Influence of Compound Ic on Sizes of Necrosis Ischemia Zones in Case of an Acute Myocardial Ischemia

The experiments are carried out with non-pedigree male rats having a weight of 250-300 g, which have been anesthetized with sodium ethaminale (40 mg/kg intraperitoneally). A myocardial infarction was modeled for animals, transferred to controlled breathing, by ligation of a descending branch of the left-hand coronary artery to a level of the lower edge of an auricula atrii.

The sizes of necrosis zones and ischemia zones have been determined within 4 hours after occlusion of the coronary artery by a differential indicator method, the principle of which is based on separate quantitative determination of Evans' blue (indicator of an ischemia zone) and red phormazane (indicator of an necrosis zone).

TABLE 4
Anti-ischemic activity of compound Ic in comparison
with other 3-oxypyridin derivatives (within 4 hours
after occlusion of the coronary artery of rats)

| | | | Ratio of necrosis | |
|---|---|---|---|---|
| | | | zone to total | Ratio of necrosis |
| Experimental | Dose | Number of | mass of the | zone to ischemia |
| conditions | mg/kg | animals | myocardium (%) | zone (%) |
| Control | — | 17 | 22 ± 2.0 | 68 ± 4.3 |
| Compound 1 | 16 | 7 | 4 ± 2.3 | 11 ± 3.3 |
| Emoxypine | 26 | 8 | 9 ± 2.4 | 32 ± 4.6 |
| Mexydole | 26 | 8 | 8 ± 1.4 | 46 ± 5.6 |
| Nicorandile | 12 | 8 | 10 ± 1.6 | 42 ± 5.4 |

As can be seen, compound Ic shows a considerably higher anti-ischemic activity than the other 3-oxypyridines.

### Example 6

### Study of Local Irritant Action of Compound Ic to Tissue of the Eye

The experimental measurements are carried out on six rabbits. All animals have been subjected to single instillation of 1% solution of compound I into the right eye and 1% solution of emoxypine into the left eye. The front section of the eye is controlled by a focal illumination method with a 20 D lens within 1 min. after dropping. The results show, that:

A reaction of the conjunctiva of the eyelids and of the eyeglobes, or the cornea is not detected in the right eyes of the animals.

A pronounced conjunctival injection of the eyeglobe and a hyperemia of the conjunctiva of the low eyelid are observed in the left eyes of 4 animals. The other two animals show a moderate conjunctival injection of the eye.

The results, thus, demonstrate, that claimed compound Ic and the salts thereof have antioxidant, geroprotective and anti-ischemic activities and do not show local irritating action on tissue of the eye.

### Example 7

The efficiency of compound Ic in traumatic hemophthalmia, on the basis of the modern ultrasonic researches of eyeglobe (eyeball) and parameters of biochemical researches of blood serum, tear liquid, liquid of the anterior chamber and eye tissue was studied.

Material and Methods:

Studies were carried out in 12 chinchilla rabbits having body weight 2.0-2.5 kg. A traumatic hemophthalmia was modeled by introduction of 0.5 to 0.7 ml of autoblood into a vitreous body through a puncture in a sclera on distance of 5 mm from a limbus, under a local anaesthesia. The basic group made with 6 rabbits (12 eyes), daily received a formulation prepared in accordance with the present invention parabulbarly (0.5 ml of 1% solution). A control group presented by 6 animals (12 eyes), daily received parabulbar emoxipin (2-ethyl-6-methyl-3-hydroxypiridin) in the same dosage. Treatment was carried out within 14 days after a trauma.

Besides the traditional ophthalmologic examination such as biomicroscopy, direct and indirect ophthalmoscopy, all the animals passed through ultrasonic scanning of an eye by means of the device “Voluson 730” (“Kretz”). The studies were carried out on the 1, 3, 7, 10 and 14^{th} day of the experiment. For the estimation of efficiency of the treatment following tests were used: density, area, volume of a hemophthalmia and opportunity (possibility) of ophthalmoscopy of an eye ground. Density of a hemophthalmia was estimated in standard (conventional) units of density (SUD), 100 SUD were accepted for the maximal value, corresponding to the ultrasonic sclera density. Depending on the degree of acoustic density of a hemophthalmia, a high (100-50 SUD), medium (50-25 SUD) and low (less than 25 SUD) densities were distinguished. To determine the hemophthalmia the area measurement in sm² was used. The volume of the intraocular hemorrhage was studied with the help of 3D modelling in the B-regimen of a grey scale and measurement in sm³.

At the ultrasonic examination of the intact rabbit's eyes, the average volume of vitreous body (1.0±0.9 sm³) and the average area of a vitreous body (1.04±0.8 sm²) were fixed. Proceeding from the obtained data, the total hemophthalmia (from 100 up to 50% of the vitreous body volume) was equated to the volume of 0.5-1.0 sm³ and of 0.5-1.0 sm², a wide-spread hemophthalmia (50-25% of volume of a vitreous) corresponded to the area of 0.5-0.25 sm³, both 0.5-0.25 sm², and the partial hemophthalmia occupied up to 0.25 sm³ and 0.25 sm² (up to 25% of the vitreous volume).

Capability of the eyeground ophthalmoscopy was estimated by a 3-mark(point) scale depending on the image sharpness (0—ophthalmoscopy is impossible (not capable), 3—details of an eyeground are clearly visible).

The materials used for biochemical examination were as follows: blood, liquid of the anterior chamber, tear liquid, tunics of eyeglobe (retina, a vitreous body). The blood was taken from the aural vein in an amount of 3 ml. Sampling of tear liquid was carried out by a microcapillary after instillation of distilled water into the conjunctival cavity. Liquid of the anterior chamber was obtained by paracentesis. The blood sampling as well as liquid of the anterior chamber and teat liquid were carried out on the 1^{st}, 7^{th} and 14^{th} day of the experiment. On the 15^{th} day of the studies, the animals were killed (pithed) by air embolism and both the eyes were enucleated. The eyes were prepared by tunic separation. Retina and vitreous body were obtained.

For estimation of activity of the preparations the concentration of products of the free radical oxidation, active in the reaction with thiobarbituric acid (TBA-active products), protein concentration, and the antioxidant activity (AOA) were determined

Results:

The data of preclinical and ultrasonic examinations at the experimental hemophthalmia in dynamics is shown in Table No. 5.

TABLE 5
Dynamics of parameters of preclinical and ultrasonic examination at the
experimental hemophthalmia in the basic (A) and control (B) groups.

| | Parameters | | | | | | | |
|---|---|---|---|---|---|---|---|---|
| | | | | | | | Capability of | |
| | | | | | Density of | | ophthalmoscopy | |
| | Volume of a | | Area of | | hemophthalmia, | | and eyeground, | |
| | hemophthalmia, sm³ | | hemophthalmia, sm² | | SUD | | points | |
| Day | A | B | A | B | A | B | A | B |
| 1^{st} | 0.53 ± 0.4 | 0.54 ± 0.8 | 0.49 ± 0.7 | 0.48 ± 0.5 | 84 ± 2.4 | 82 ± 1.4 | 1.5 | 1.5 |
| 3^{rd} | 0.78 ± 0.2 | 0.81 ± 0.8 | 0.74 ± 0.9 | 0.76 ± 0.1 | 72 ± 3.1 | 76 ± 2.6 | 0-0.5 | 0-0.5 |
| 7^{th} | 0.51 ± 0.6 | 0.7 ± 0.1 | 0.47 ± 0.4 | 0.68 ± 0.9 | 49 ± 1.4 | 65 ± 2.1 | 1.5 | 0.8 |
| 10^{th} | 0.24 ± 0.1 | 0.53 ± 0.4 | 0.2 ± 0.1 | 0.5 ± 0.2 | 23 ± 1.1 | 48 ± 1.5 | 2.2 | 1.6 |
| 14^{th} | 0.15 ± 0.2 | 0.4 ± 0.7 | 0.11 ± 0.7 | 0.39 ± 0.4 | 13 ± 0.6 | 32 ± 0.8 | 2.8 | 2.0 |

In the first day of experiment, the parameters of ultrasonic and clinical examination were practically identical in all groups: the density of hemophthalmia in average made from 81 up to 85 SUD, the area—0.52-0.55 sm², the volume 0.47-0.5 sm³, the capability of ophthalmoscopy of an eyeground was corresponded to 1-2 points. Thus, in all animals the intraocular hemorrhage which complicated the capability of ophthalmoscopy of an eyeground was marked rather small in volume, but essential in density.

By the 3^{rd} day of studies the blood was regularly distributed in a vitreous body, thus the density of hemophthalmia was decreased a little. The area and the volume of a hemorrhage were corresponded to criteria of the total hemophthalmia (about 80% of a vitreous body), the density remained high (more than 70 SUD), the eye ground practically was not looked through (seen) (0-0.5 points). Since from the 3^{rd} day of experiment the tendency to the faster resorption of hemorrhage of rabbits of the basic group was outlined, however, no statistically significant difference of ultrasonic examination was observed. To characterize clinical current of the given period it is necessary to refer the more expressed reaction of an eye to a trauma in animals of the control group in comparison with the experienced group (lacrimation, photophobia, injection of an eyeglobe, liquid opalescence of the anterior chamber).

By the 7^{th} day, the difference of preclinical and ultrasonic parameters of a hemophthalmia became more noticeable. In the basic group, the area of a hemophthalmia was decreased up to 0.51±0.6 sm², volume up to 0.47±0.4 sm³, density up to 49±1.4 SUD; capability of the eyeground ophthalmoscopy was equal to 1.5 points. In the basic group the wide-spread hemophthalmia of average density was observed. In the control group, the tendency to resorption of a hemorrhage was less expressed: the area of a hemophthalmia was 0.7±0.1 sm², volume 0.68±0.9 sm³, density 65±2.1 SUD, capability of the eyeground ophthalmoscopy was 1 point. Thus, in animals of the control group the clinical picture practically did not changed.

By the 10^{th} day, in the basic group the hemophthalmia was completely resolved in two animals, in other cases marked a noticeable decrease of the area (0.24±0.1 sm²), volume (0.2±0.1 sm³) and density (23±1.1 SUD) of hemorrhage, the eyeground (2.2 points) was well seen. The intraocular hemorrhage corresponded to a partial hemophthalmia with a low density. In the control group the effect of treatment was less expressed: the area of a hemophthalmia averaged to 0.53±0.4 sm², volume 0.5±0.2 sm³, density 48±1.5 SUD, capability of the eyeground ophthalmoscopy was equal to 1.8 points. On the 10^{th} day of the studies, some rabbits receiving emoxipin had the tendency of sheet-anchor formation in a vitreous body.

On the 14^{th} day the hemophthalmia was completely resolved in 50% of animals in the basic group, the area, volume and density (0.15±0.2 sm², 0.11±0.7 sm³ and 13±0.6 SUD, accordingly) of hemorrhage were considerably decreased, the eyeground (2.6 points) was well seen in the other animals. In the control group, the result of the treatment appeared less expressed: the area of a hemophthalmia was 0.47±0.7 sm², volume 0.39±0.4 sm³, density 32±0.8 SUD, the ophthalmoscopy picture of an eyeground was not clear (2 points). In the control group, no noticeable changes were obtained in comparison with the 10^{th} day; the phenomena of fibrosis and sheet-anchor formation in a vitreous body were marked in 30% of animals.

Table No. 6 shows biochemical parameters in blood serum (BS), tear liquid (TL) and the anterior chamber liquid (ACL) at an experimental hemophthalmia in the basic (A) and the control (B) groups.

TABLE 6
Biochemical parameters

| | Protein concentration | | Concentration of TBA- | | | |
|---|---|---|---|---|---|---|
| | (mg/ml) | | active products (nmol/ml) | | AOA (relative unit) | |
| | A | B | A | B | A | B |
| 1^{st} day | | | | | | |
| BS | 90.0 ± 6.0 | 85.2 ± 0.9 | 1.29 ± 0.13 | 2.53 ± 0.28 | 1.7 ± 0.3 | 1.5 ± 0.2 |
| TL | 0.32 ± 0.2 | 0.35 ± 0.1 | 0.14 ± 0.01 | 0.16 ± 0.02 | 0.23 ± 0.02 | 0.13 ± 0.03 |
| ACL | 3.1 ± 1.2 | 3.9 ± 1.0 | 0.97 ± 0.18 | 1.6 ± 0.3 | 0.4 ± 0.01 | 0.33 ± 0.02 |
| 7^{th} day | | | | | | |
| BS | 82.4 ± 1.3 | 103.0 ± 5.0 | 1.17 ± 0.05 | 2.4 ± 0.09 | 2.4 ± 0.02 | 1.8 ± 0.07 |
| TL | 0.7 ± 0.2 | 0.91 ± 0.25 | 0.13 ± 0.05 | 0.46 ± 0.07 | 0.24 ± 0.02 | 0.18 ± 0.05 |
| ACL | 4.4 ± 1.2 | 10.0 ± 3.0 | 0.8 ± 0.5 | 2.2 ± 0.4 | 0.8 ± 0.07 | 0.55 ± 0.04 |
| 14^{th} day | | | | | | |
| BS | 47.0 ± 3.0 | 73.0 ± 2.0 | 0.9 ± 0.15 | 1.6 ± 0.2 | 1.9 ± 0.7 | 1.3 ± 0.3 |
| TL | 0.6 ± 0.2 | 0.74 ± 0.14 | 0.04 ± 0.07 | 0.3 ± 0.2 | 0.08 ± 0.02 | 0.06 ± 0.03 |
| ACL | 4.1 ± 1.1 | 5.5 ± 1.0 | 0.6 ± 0.2 | 2.2 ± 0.3 | 0.5 ± 0.05 | 0.33 ± 0.06 |
| Retina | | | | | 0.4 ± 0.07 | 0.23 ± 0.08 |
| Vitreous | | | | | 3.5 ± 0.9 | 1.6 ± 0.7 |
| body | | | | | | |

From the mentioned data it is evident that the protein concentration in the 1^{st} day of experiment, and AOA in all biological liquids practically did not varied in the groups. Concentration of TBA-active products in the blood serum had no essential differences either, however, in the tear and anterior chamber liquid of the rabbits of the basic group, the concentration of TBA-active products was lower than that in the rabbits of the control group.

The greatest difference in biochemical parameters observed on the 7-th day of experiment and coincided with the beginning of the active hemorrhage resorption in rabbits of the basic group. In animals of the basic group AOA was in the average 1.5 times higher than in the control group in all measured substrata: in the blood serum 2.4±0.02-1.8±0.07/100 mcl, tear liquid 0.24±0.02-0.18±0.05/200 mcl and in the anterior chamber liquid 0.8±0.07-0.55±0.04/100 mcl. On the contrary, concentration of TBA-active products was grown in the control group (2.4±0.09 nmol/ml in the blood serum, 0.46±0.07 nmol/ml in the tear liquid, 2.2±0.4 nmol/ml in the anterior chamber liquid), and was reduced in the basic group (1.17±0.05 nmol/ml in the blood serum, 0.13±0.05 nmol/ml in the tear liquid, 0.8±0.5 nmol/ml in the anterior chamber liquid). Similar changes were observed at examination of protein concentration in the blood serum in rabbits of the control (103.0±5.0 mg/ml) and the basic (82.4±1.3 mg/ml) groups. In all animals the protein concentration was grown in the tear and the anterior chamber liquid on the 7^{th} day.

By the 14^{th} day of experiment, statistically significant differences in the biochemical parameters were marked. In both groups, AOA was insignificantly reduced, however the higher parameters were in the animals receiving formulation prepared in accordance with the present invention (1.9±0.7/100 mcl in the blood serum, 0.08±0.02/200 mcl in the tear liquid, 0.5±0.05/100 mcl in the anterior chamber liquid), and the lower parameters were in the animals receiving emoxipin (1.3±0.03/100 mcl in the blood serum, 0.06±0.03/200 mcl in the tear liquid, 0.33±0.06/100 mcl in the anterior chamber liquid). Concentration of TBA-active products was also decreased in all rabbits, thus in the basic group their values were lower, than in the control group (0.9±0.15-1.6±0.2 nmol/ml in the blood serum, 0.04±0.07-0.3±0.2 nmol/ml in the tear liquid, 0.6±0.2-2.2±0.3 nmol/ml in the anterior chamber liquid, accordingly). Similar changes were marked at the examination of parameters of protein concentration in both the groups of animals.

In AOA studies the augmentation of the given parameter was observed in 2 times in the eye tunics, a retina and a vitreous body, in rabbits of the basic group in comparison with the control group.

Conclusions:

1. Compound Ic prepared in accordance with the present invention renders a positive effect on the resorptive processes in a vitreous body at the experimental traumatic hemophthalmia.

2. On the basis of parameters of clinical and ultrasonic examination of eyes of the experimental animals at the traumatic hemophthalmia in dynamics, it is established that efficiency of the compound Ic prepared in accordance with the present invention is much higher in comparison with emoxipin.

3. Results of biochemical studies of the blood serum, tear liquid, the anterior chamber liquid and eye tissues (in vivo) shows that the compound Ic prepared in accordance with the present invention has higher antioxidant activity than emoxipin.

The comparative evaluation of antioxidant, antiradical geroprotective and anti-ischemic properties of compound Ic and drugs, widely used in medical practice being mexydole and emoxypine indicates a substantially higher efficiency and a lower toxicity of the claimed agent, and also indicates the prospectivity of the use of said agent in ophthalmic and cardiologic practice and other fields of medicine.

### Example 8

### Oral Formulations

### Example 8A

### 3-hydroxy-2,4,6-trimethylpyridine succinate capsule

A capsule of 3-hydroxy-2,4,6-trimethylpyridine succinate was formulated as follows:

| | | |
|---|---|---|
| 3-hydroxy-2,4,6-trimethylpyridine succinate | 100.0 | mg |
| Microcrystalline cellulose | 95.0 | mg |
| Lactose | 30.0 | mg |
| Maize starch | 89.7 | mg |
| Maize starch (for paste) | 20.0 | mg |
| Methyl paraben | 0.2 | mg |
| Propyl paraben | 0.1 | mg |
| Sodium starch glycollate | 8.0 | mg |
| Purified talc | 2.0 | mg |
| Magnesium stearate | 2.0 | mg |
| Colloidal anhydrous silica | 3.0 | mg |

| | |
|---|---|
| Purified water | Q.S. |

Microcrystalline cellulose, lactose and maize starch were mixed together in a blender and then granulated using starch paste containing methyl paraben and propyl paraben. The granules were dried, passed through No. 30 stainless steel screen to obtain suitable particle size distribution. The granules were blended with 3-hydroxy-2,4,6-trimethylpyridine succinate, sodium starch glycollate talc, colloidal anhydrous silica and magnesium stearate. The prepared blended mass was filled in size ‘0’ opaque capsules.

### Example 8B

### 3-hydroxy-2,4,6-trimethylpyridine succinate capsule

A capsule of 3-hydroxy-2,4,6-trimethylpyridine succinate was formulated as follows:

| | | |
|---|---|---|
| 3-hydroxy-2,4,6-trimethylpyridine succinate | 100.0 | mg |
| Microcrystalline cellulose direct compression grade | 90.0 | mg |
| Lactose direct compression grade | 90.0 | mg |
| Sodium starch glycollate | 13.0 | mg |
| Purified talc | 2.0 | mg |
| Magnesium stearate | 2.0 | mg |
| Colloidal anhydrous silica | 3.0 | mg |

Lubricated blend of 3-hydroxy-2,4,6-trimethylpyridine succinate was prepared with microcrystalline cellulose, lactose, sodium starch glycollate, talc, colloidal anhydrous silica and magnesium stearate. The prepared blended mass was then filled in size ‘0’ opaque capsules.

### Example 8C

### 3-hydroxy-2,4,6-trimethylpyridine succinate tablet

A tablet of 3-hydroxy-2,4,6-trimethylpyridine succinate was formulated as follows:

3-hydroxy-2,4,6-trimethylpyridine succinate 100.0 mg

| | | |
|---|---|---|
| Microcrystalline cellulose | 95.0 | mg |
| Lactose | 30.0 | mg |
| Maize starch | 90.0 | mg |
| Maize starch (For Paste) | 20.0 | mg |
| Crospovidone | 7.0 | mg |
| Purified talc | 2.0 | mg |
| Magnesium stearate | 3.0 | mg |
| Colloidal anhydrous silica | 3.0 | mg |

| | |
|---|---|
| Purified water | Q.S. |

3-hydroxy-2,4,6-trimethylpyridine succinate, microcrystalline cellulose, lactose and maize starch were granulated using starch paste. The granules were dried, screened through stainless steel screen No. 30 and mixed with a blend of crospovidone, talc, colloidal anhydrous silica and magnesium stearate. The blended mass was then compressed.

### Example 8D

### 3-hydroxy-2,4,6-trimethylpyridine succinate tablet

A film coated tablet of 3-hydroxy-2,4,6-trimethylpyridine succinate was formulated as follows:

| | | |
|---|---|---|
| 3-hydroxy-2,4,6-trimethylpyridine succinate | 100.0 | mg |
| Microcrystalline cellulose direct compression grade | 95.0 | mg |
| Dicalcium phosphate compression grade | 90.0 | mg |
| Croscarmellose sodium | 7.0 | mg |
| Purified Talc | 2.0 | mg |
| Magnesium stearate | 3.0 | mg |
| Colloidal anhydrous silica | 3.0 | mg |

Lubricated blend of 3-hydroxy-2,4,6-trimethylpyridine succinate was prepared with microcrystalline cellulose, diclacium phosphate, crosscarmellose sodium talc, colloidal anhydrous silica and magnesium stearate. The blended mass was then compressed to form the tablet. Further, the tablet was coated with coating solution containing hydroxypropylmethylcellulose, titanium dioxide, propylene glycol and iron oxide yellow in purified water.

### Example 8E

### 3-hydroxy-2,4,6-trimethylpyridine succinate tablet

A tablet of 3-hydroxy-2,4,6-trimethylpyridine succinate was formulated as follows:

| | | |
|---|---|---|
| 3-hydroxy-2,4,6-trimethylpyridine succinate | 100.0 | mg |
| Microcrystalline cellulose | 95.0 | mg |
| Lactose | 30.0 | mg |
| Maize starch | 90.0 | mg |
| Maize starch (for paste) | 20.0 | mg |
| Crospovidone | 8.0 | mg |
| Sodium saccharin | 3.5 | mg |
| Orange flavor | 1.5 | mg |
| Purified talc | 2.0 | mg |
| Magnesium stearate | 2.0 | mg |
| Colloidal anhydrous silica | 3.0 | mg |

| | |
|---|---|
| Purified Water | Qs |

Microcrystalline cellulose, lactose, maize starch and 3-hydroxy-2,4,6-trimethylpyridine succinate were granulated using starch paste. The granules were dried, screened through stainless steel screen No. 30 and then blended the dried granules with crosspovidone, sodium saccharin, orange flavor, talc, colloidal anhydrous silica and magnesium stearate. Finally the blended mass was compressed.

### Example 8F

### 3-hydroxy-2,4,6-trimethylpyridine succinate tablet

A tablet of 3-hydroxy-2,4,6-trimethylpyridine succinate was formulated as follows:

| | | |
|---|---|---|
| 3-hydroxy-2,4,6-trimethylpyridine succinate | 100.0 | mg |
| Microcrystalline cellulose direct compression grade | 95.0 | mg |
| Mannitol direct compression grade | 30.0 | mg |
| Pregelatinsed maize starch | 50.0 | mg |
| Croscarmellose sodium | 8.0 | mg |
| Sodium saccharin | 3.5 | mg |
| Sorbitol | 2.0 | mg |
| Orange flavor | 3.5 | mg |
| Purified talc | 2.0 | mg |
| Magnesium stearate | 2.0 | mg |
| Colloidal anhydrous silica | 3.0 | mg |

All the ingredients were sifted through stainless steel screen No. 30 and mixed in a blender. The blended mass was then compressed.

### Example 8G

### 3-hydroxy-2,4,6-trimethylpyridine succinate powder

A powder of 3-hydroxy-2,4,6-trimethylpyridine succinate was formulated as follows:

| | | |
|---|---|---|
| 3-hydroxy-2,4,6-trimethylpyridine succinate | 100 | mg |
| Sucrose | 95.0 | mg |
| Maize starch | 50 | mg |
| Povidone | 10 | mg |
| Citric acid | 3.0 | mg |
| Sodium citrate | 2.0 | mg |
| Sodium saccharin | 3.5 | mg |
| Sorbitol | 2.0 | mg |
| Orange flavor | 3.5 | mg |
| Colloidal anhydrous silica | 3.0 | mg |

| | |
|---|---|
| Water | QS |

The granules of sucrose, maize starch and 3-hydroxy-2,4,6-trimethylpyridine succinate were prepared using aqueous povidone solution. The prepared granules were dried and screened through stainless steel screen No. 20. The granules were further blended with sodium saccharin, orange flavor, sorbitol, sodium citrate, citric acid and colloidal anhydrous silica.

### Example 8H

### 3-hydroxy-2,4,6-trimethylpyridine succinate effervescent tablet

An effervescent tablet of 3-hydroxy-2,4,6-trimethylpyridine succinate was formulated as follows:

| | | |
|---|---|---|
| 3-hydroxy-2,4,6-trimethylpyridine succinate | 100.0 | mg |
| Sorbitol instant | 43.5 | mg |
| Xylitol | 43.5 | mg |
| Citric acid anhydrous | 140.0 | mg |
| Sodium bicarbonate | 155.0 | mg |
| Sodium saccharin | 5.0 | mg |

| | |
|---|---|
| Flavor | Qs |
| Color | Qs |
| Magnesium Stearate | Qs |

3-Hydroxy-2,4,6-trimethylpyridine succinate, sorbitol, xylitol, citric acid, sodium bicarbonate, sodium saccharin, flavor, color and lubricant were blended in a blender. The blended mass was compressed.

### Example 8I

### 3-hydroxy-2,4,6-trimethylpyridine succinate powder (dry syrup)

Powder of 3-hydroxy-2,4,6-trimethylpyridine succinate was formulated as follows:

| | | |
|---|---|---|
| 3-hydroxy-2,4,6-trimethylpyridine succinate | 2.0% | w/v |
| Sucrose | 65.0% | w/v |
| Mannitol | 20.0% | w/v |
| Sodium benzoate | 0.2% | w/v |
| Citric acid | 0.3% | w/v |
| Sodium citrate | 0.3% | w/v |
| Sodium saccharin | 0.5% | w/v |
| Orange flavor | 0.5% | w/v |
| Xanthan gum | 0.2% | w/v |

All the ingredients were passed through S.S. sieve No. 30 and blended together in a blender. The resultant dry syrup was filled in a bottle.

### Example 8J

### 3-hydroxy-2,4,6-trimethylpyridine succinate liquid

A liquid of 3-hydroxy-2,4,6-trimethylpyridine succinate was formulated as follows:

| | | |
|---|---|---|
| 3-hydroxy-2,4,6-trimethylpyridine succinate | 2.0% | w/v |
| Sucrose | 75.0% | w/v |
| Non crystallizing sorbitol | 10.0% | w/v |
| Methyl paraben | 0.2% | w/v |
| Propyl paraben | 0.02% | w/v |
| Citric acid | 0.3% | w/v |
| Sodium citrate | 0.3% | w/v |
| Sodium saccharin | 0.5% | w/v |

| | |
|---|---|
| Orange flavor | Qs |
| Color | Qs |
| Water | Qs |

Methyl paraben and propyl paraben were dissolved in hot water. To this sucrose was added to obtain syrup. 3-hydroxy-2,4,6-trimethylpyridine succinate, citric acid, sodium citrate and color were added to the syrup with continuous stirring. Flavor was added in the final step. The pH of the syrup in was maintained in between 3 to 6.

### Example 9

### Ophthalmic Formulations

### Example 9A

### 3-hydroxy-2,4,6-trimethylpyridine succinate solution

A solution of 3-hydroxy-2,4,6-trimethylpyridine succinate was formulated as follows:

| | |
|---|---|
| 3-Hydroxy-2,4,6-trimethylpyridine succinate | 1.0% |
| Benzylkonium chloride | 0.01% |
| Hydrochloric acid | QS |
| Sodium hydroxide | QS |
| Potassium monobasic phosphate | QS |
| Dibasic sodium phosphate | QS |
| HPMC 15cps | 0.50% |
| Water for injection | QS |

3-hydroxy-2,4,6-trimethylpyridine succinate and other ingredients were dissolved in water for injection. The pH of the solution was adjusted to 6. The resultant solution was sterilized and aseptically filled into vials.

### Example 9B

A solution of 3-hydroxy-2,4,6-trimethylpyridine succinate was formulated as follows:

| | |
|---|---|
| 3-hydroxy-2,4,6-trimethylpyridine succinate | 1.0% |
| Benzalkonium chloride | 0.01% |
| Hydrochloric acid | QS |
| Sodium hydroxide | QS |
| Sodium benzoate | 0.15% |
| Potassium monobasic phosphate | QS |
| Dibasic sodium phosphate | QS |
| HPMC 15cps | 0.50% |
| Water for injection | QS |

### Example 9C

A solution of 3-hydroxy-2,4,6-trimethylpyridine succinate was formulated as follows:

| | |
|---|---|
| 3-hydroxy-2,4,6-trimethylpyridine succinate | 1.0% |
| Benzalkonium chloride | 0.01% |
| Sodium chloride | 0.06% |
| Hydrochloric acid | QS |
| Sodium hydroxide | QS |
| Potassium monobasic phosphate | QS |
| Dibasic sodium phosphate | QS |
| Water for injection | QS |

### Example 9D

A solution of 3-hydroxy-2,4,6-trimethylpyridine succinate was formulated as follows:

| | |
|---|---|
| 3-hydroxy-2,4,6-trimethylpyridine succinate | 1.0% |
| Benzalkonium chloride | 0.01% |
| EDTA | 0.05% |
| Mannitol | 2.9% |
| Sodium acetate | QS |
| Glacial acetic acid | QS |
| Hydrochloric acid | QS |
| Sodium hydroxide | QS |
| Water for injection | QS |

### Example 9E

A solution of 3-hydroxy-2,4,6-trimethylpyridine succinate was formulated as follows:

| | |
|---|---|
| 3-hydroxy-2,4,6-trimethylpyridine succinate | 1.0% |
| Benzalkonium chloride | 0.01% |
| EDTA | 0.05% |
| Mannitol | 2.0% |
| Sodium chloride | 0.2% |
| Hydrochloric acid | Qs |
| Sodium hydroxide | Qs |
| HPMC (15 cps) | 0.50% |
| Water for injection | QS |

### Example 9F

A gel of 3-hydroxy-2,4,6-trimethylpyridine succinate was formulated as follows:

| | |
|---|---|
| 3-hydroxy-2,4,6-trimethylpyridine succinate | 1.0% |
| Carbopol 934P | 2% |
| Triethanolamine | QS |
| Benzalkonium chloride | QS |
| Water for injection | QS |

Carbopol 934P was dispersed in water for injection and stirred for 1 hour. Resultant dispersion was kept for soaking for 10 hours to form a clear gel. To this 3-hydroxy-2,4,6-trimethylpyridine succinate dissolved in water for injection was added with continuous stirring. Further, benzalkonium chloride was added and stirred for 10 minutes. Finally, triethanolamine was added and pH of the gel was adjusted to 5.9. The formulation was sterilized and aseptically filled into the tubes.

### Example 9G

An ointment of 3-hydroxy-2,4,6-trimethylpyridine succinate was formulated as follows:

3-hydroxy-2,4,6-trimethylpyridine succinate 1.0%

| | |
|---|---|
| White soft paraffin | 80.0% |
| Liquid paraffin | 5.0% |
| Cetsteryl alcohol | 5.0% |
| Hard paraffin | 5.0% |
| Benzalkonium chloride | QS |
| Buffer | QS |
| Water for injection | QS |

The ointment base was prepared by melting together white soft paraffin, liquid paraffin, cetsteryl alcohol and hard paraffin. To this base, 3-hydroxy-2,4,6-trimethylpyridine succinate and benzalkonium chloride dissolved in water for injection were added and homogeneously blended. The pH of the ointment was adjusted to 5.8. The ointment was sterilized and aseptically filled into the tubes.

### Example 9H

An ointment of 3-hydroxy-2,4,6-trimethylpyridine succinate was formulated as follows:

| | |
|---|---|
| 3-Hydroxy-2,4,6-trimethylpyridine succinate | 1.0% |
| White soft paraffin | 65.0% |
| Liquid paraffin | 10.0% |
| Cetsteryl alcohol | 7.0% |
| Hard paraffin | 12.0% |
| Benzalkonium chloride | QS |
| Buffer | QS |
| Water for injection | QS |

### Example 9I

An ointment of 3-hydroxy-2,4,6-trimethylpyridine succinate was formulated as follows:

| | |
|---|---|
| 3-Hydroxy-2,4,6-trimethylpyridine succinate | 1.0% |
| White Soft Paraffin | 65.0% |
| Hard paraffin | 5.0% |
| Liquid Paraffin | 25.0% |
| Preservative | QS |
| Buffer | QS |
| Water for injection | QS |

### Example 9J

An ointment of 3-hydroxy-2,4,6-trimethylpyridine succinate was formulated as follows:

| | |
|---|---|
| 3-Hydroxy-2,4,6-trimethylpyridine succinate | 1.0% |
| White Soft Paraffin | 67.0% |
| Liquid Paraffin | 29.0% |
| Preservative | QS |
| Buffer | QS |
| Water for injection | QS |

### Example 10

### Injection Formulations

### Example 10A

| | |
|---|---|
| 3-hydroxy-2,4,6-trimethylpyridine succinate | 10.0 mg/ml |
| 10% aqueous solution of hydrochloric acid | QS |
| Water for injection | upto 1 ml |

3-hydroxy-2,4,6-trimethylpyridine succinate was dissolved in water for injection with continuous stirring under inert gas. The pH of the solution was adjusted to 2.9 with 10% aqueous solution of hydrochloric acid. The solution was diluted with sterile water for injection to achieve required concentration of 10 mg per ml. Further, the resultant solution was sterilized by sterile filtration and autoclaving. The sterile formulation was aseptically filled into ampoule.

### Example 10B

| | |
|---|---|
| 3-hydroxy-2,4,6-trimethylpyridine maleate | 10.0 mg/ml |
| 10% aqueous solution of hydrochloric acid | QS |
| Water for injection | upto 1 ml |

The process of example 10A was repeated except that 3-hydroxy-2,4,6-trimethylpyridine maleate was employed as the active ingredient.

### Example 10C

| | |
|---|---|
| 3-hydroxy-2,4,6-trimethylpyridine tartrate | 10.0 mg/ml |
| 10% aqueous solution of hydrochloric acid | QS |
| water for injection | upto 1 ml |

The process of example 10A was repeated except that 3-hydroxy-2,4,6-trimethylpyridine tartrate was employed as the active ingredient.

### Example 10D

| | |
|---|---|
| 3-hydroxy-2,4,6-trimethylpyridine oxalate | 10.0 mg/ml |
| 10% aqueous solution of hydrochloric acid | QS |
| water for injection | upto 1 ml |

The process of example 10A was repeated except that 3-hydroxy-2,4,6-trimethylpyridine oxalate was employed as the active ingredient.

### Example 10E

| | |
|---|---|
| 3-hydroxy-2,4,6-trimethylpyridine succinate | 50.0 mg/ml |
| 10% aqueous solution of hydrochloric acid | QS |
| water for injection | upto 1 ml |

3-hydroxy-2,4,6-trimethylpyridine succinate was dissolved in water for injection with continuous stirring under inert gas. The pH of the solution was adjusted to 2.9 with 10% aqueous solution of hydrochloric acid. The solution was diluted with sterile water for injection to achieve required concentration of 50 mg per ml. Further, the resultant solution was sterilized by sterile filtration and autoclaving. The sterile formulation was aseptically filled into the ampoule.

### Example 10F

| | |
|---|---|
| 3-hydroxy-2,4,6-trimethylpyridine maleate | 50.0 mg/ml |
| 10% aqueous solution of hydrochloric acid | QS |
| water for injection | upto 1 ml |

The process of example 10E was repeated except that 3-hydroxy-2,4,6-trimethylpyridine maleate was employed as the active ingredient.

### Example 10G

| | |
|---|---|
| 3-Hydroxy-2,4,6-trimethylpyridine tartarate | 50.0 mg/ml |
| 10% aqueous solution of hydrochloric acid | QS |
| water for injection | upto 1 ml |

The process of example 10E was repeated except that 3-hydroxy-2,4,6-trimethylpyridine tartarate was employed.

### Example 10H

| | |
|---|---|
| 3-Hydroxy-2,4,6-trimethylpyridine oxalate | 50.0 mg/ml |
| 10% aqueous solution of hydrochloric acid | QS |
| water for injection | upto 1 ml |

The process of example 10E was repeated except that 3-hydroxy-2,4,6-trimethylpyridine oxalate was employed as the active ingredient.

### Example 10I

| | |
|---|---|
| 3-hydroxy-2,4,6-trimethylpyridine succinate | 10.0 mg/ml |
| 10% aqueous solution of hydrochloric acid | QS |
| water for injection | upto 1 ml |

3-hydroxy-2,4,6-trimethylpyridine succinate was dissolved in water for injection with continuous stirring under inert gas. The pH of the solution was adjusted to 4.8 with 10% aqueous solution of hydrochloric acid. The solution was diluted with sterile water for injection to achieve required concentration of 10 mg per ml. Further, the resultant solution was sterilized by sterile filtration and autoclaving. The sterile formulation was aseptically filled into the ampoule.

### Example 10J

| | |
|---|---|
| 3-hydroxy-2,4,6-trimethylpyridine maleate | 10.0 mg/ml |
| 10% aqueous solution of hydrochloric acid | QS |
| water for injection | upto 1 ml |

The process of example 10I was repeated except that 3-hydroxy-2,4,6-trimethylpyridine maleate was employed as the active ingredient.

### Example 10K

| | |
|---|---|
| 3-hydroxy-2,4,6-trimethylpyridine tartarate | 10.0 mg/ml |
| 10% aqueous solution of hydrochloric acid | QS |
| water for injection | upto 1 ml |

The process of example 10I was repeated except that 3-hydroxy-2,4,6-trimethylpyridine tartarate was employed as the active ingredient.

### Example 10L

| | |
|---|---|
| 3-hydroxy-2,4,6-trimethylpyridine oxalate | 10.0 mg/ml |
| 10% aqueous solution of hydrochloric acid | QS |
| water for injection | upto 1 ml |

The process of example 101 was repeated except that 3-hydroxy-2,4,6-trimethylpyridine oxalate was employed.

### Example 10M

| | |
|---|---|
| 3-hydroxy-2,4,6-trimethylpyridine succinate | 50.0 mg/ml |
| 10% aqueous solution of hydrochloric acid | QS |
| water for injection | upto 1 ml |

3-hydroxy-2,4,6-trimethylpyridine succinate was dissolved in water for injection with continuous stirring under inert gas. The pH of the solution was adjusted to 4.7 with 10% aqueous solution of hydrochloric acid. The solution was diluted with sterile water for injection to achieve required concentration of 50 mg per ml. Further the resultant solution was sterilized by sterile filtration and autoclaving. The sterile formulation was aseptically filled into the ampoule.

### Example 10N

| | |
|---|---|
| 3-hydroxy-2,4,6-trimethylpyridine maleate | 50.0 mg/ml |
| 10% aqueous solution of hydrochloric acid | QS |
| water for injection | upto 1 ml |

The process of example 10M was repeated except that 3-hydroxy-2,4,6-trimethylpyridine maleate was employed as the active ingredient.

### Example 10_O

| | |
|---|---|
| 3-Hydroxy-2,4,6-trimethylpyridine tartarate | 50.0 mg/ml |
| 10% aqueous solution of hydrochloric acid | QS |
| water for injection | upto 1 ml |

The process of example 10M was repeated except that 3-hydroxy-2,4,6-trimethylpyridine tartarate was employed as the active ingredient.

### Example 10P

| | |
|---|---|
| 3-Hydroxy-2,4,6-trimethylpyridine oxalate | 50.0 mg/ml |
| 10% aqueous solution of hydrochloric acid | QS |
| water for injection | upto 1 ml |

The process of example 10M was repeated except that 3-hydroxy-2,4,6-trimethylpyridine oxalate was employed as the active ingredient.

### Example 10Q

| | |
|---|---|
| 3-hydroxy-2,4,6-trimethylpyridine succinate | 10.0 mg/ml |
| benzalkonium chloride | 0.05% |
| 10% aqueous solution of hydrochloric acid | QS |
| water for injection | up to 1 ml |

3-hydroxy-2,4,6-trimethylpyridine succinate and benzalkonium chloride was dissolved in water for injection with continuous stirring under inert gas. The pH of the solution was adjusted to 4.7 with 10% aqueous solution of hydrochloric acid. The solution was diluted with sterile water for injection to achieve required concentration of 10 mg per ml. Further, the resultant solution was sterilized by sterile filtration and autoclaving. The sterile formulation was aseptically filled into the ampoule.

### Example 10R

| | |
|---|---|
| 3-hydroxy-2,4,6-trimethylpyridine maleate | 10.0 mg/ml |
| benzalkonium chloride | 0.05% |
| 10% aqueous solution of hydrochloric acid | QS |
| water for injection | up to 1 ml |

The process of example 10Q was repeated except that 3-hydroxy-2,4,6-trimethylpyridine maleate was employed as the active ingredient.

### Example 10S

| | |
|---|---|
| 3-hydroxy-2,4,6-trimethylpyridine tartarate | 10.0 mg/ml |
| benzalkonium chloride | 0.05% |
| 10% aqueous solution of sodium hydroxide | QS |
| water for injection | up to 1 ml |

3-hydroxy-2,4,6-trimethylpyridine tartarate and benzalkonium chloride was dissolved in water for injection with continuous stirring under inert gas. The pH of the solution was adjusted to 4.7 with 10% aqueous solution of sodium hydroxide. The solution was diluted with sterile water for injection to achieve required concentration of 10 mg per ml. Further, the resultant solution was sterilized by sterile filtration and autoclaving. The sterile formulation was aseptically filled into the ampoule.

### Example 10T

| | |
|---|---|
| 3-hydroxy-2,4,6-trimethylpyridine oxalate | 10.0 mg/ml |
| benzalkonium chloride | 0.05% |
| 10% aqueous solution of sodium hydroxide | QS |
| water for injection | up to 1 ml |

The process of example 10S was repeated except that 3-hydroxy-2,4,6-trimethylpyridine oxalate was employed as the active ingredient.

### Example 10U

| | |
|---|---|
| 3-hydroxy-2,4,6-trimethylpyridine succinate | 50.0 mg/ml |
| benzalkonium chloride | 0.05% |
| 10% aqueous solution of hydrochloric acid | QS |
| water for injection | up to 1 ml |

3-hydroxy-2,4,6-trimethylpyridine succinate and benzalkonium chloride was dissolved in water for injection with continuous stirring under inert gas. The pH of the solution was adjusted to 4.7 with 10% aqueous solution of hydrochloric acid. The solution was diluted with sterile water for injection to achieve required concentration of 10 mg per ml. Further, the resultant solution was sterilized by sterile filtration and autoclaving. The sterile formulation was aseptically filled into the ampoule.

### Example 10V

| | |
|---|---|
| 3-hydroxy-2,4,6-trimethylpyridine maleate | 50.0 mg/ml |
| benzalkonium chloride | 0.05% |
| 10% aqueous solution of hydrochloric acid | QS |
| water for injection | up to 1 ml |

The process of example 10U was repeated except that 3-hydroxy-2,4,6-trimethylpyridine maleate was employed as the active ingredient.

### Example 10W

| | |
|---|---|
| 3-hydroxy-2,4,6-trimethylpyridine Tartarate | 50.0 mg/ml |
| benzalkonium chloride | 0.05% |
| 10% aqueous solution of sodium hydroxide | QS |
| water for injection | up to 1 ml |

3-hydroxy-2,4,6-trimethylpyridine tartarate and benzalkonium chloride was dissolved in water for injection with continuous stirring under inert gas. The pH of the solution was adjusted to 4.7 with 10% aqueous solution of sodium hydroxide. The solution was diluted with sterile water for injection to achieve required concentration of 10 mg per ml. Further, the resultant solution was sterilized by sterile filtration and autoclaving. The sterile formulation was aseptically filled into the ampoule.

### Example 10X

| | |
|---|---|
| 3-hydroxy-2,4,6-trimethylpyridine oxalate | 50.0 mg/ml |
| benzalkonium chloride | 0.05% |
| 10% aqueous solution of Sodium Hydroxide | QS |
| Water for injection | up to 1 ml |

The process of example 10W was repeated except that 3-hydroxy-2,4,6-trimethylpyridine oxalate was employed as the active ingredient.

While considerable emphasis has been placed herein on the specific ingredients of the preferred formulation, it will be appreciated that many additional ingredients can be added and that many changes can be made in the preferred formulation without departing from the principles of the invention. These and other changes in the preferred formulation of the invention will be apparent to those skilled in the art from the disclosure herein, whereby it is to be distinctly understood that the foregoing descriptive matter is to be interpreted merely as illustrative of the invention and not as a limitation.

1. A medicinal formulation comprising a bioactive selected from the group consisting of 3-hydroxy-2,4,6 trimethylpyridine and pharmaceutically acceptable salts, esters, derivatives and polymorphs of 3-hydroxy-2,4,6 trimethylpyridine in an amount of about 0.05 to about 50% of the mass of the formulation and at least one pharmaceutically acceptable excipient selected from the group consisting of diluents, disintegrants, lubricants, glidents, binders, surfactants, solvents, coating polymers, effervescent agents, sweeteners, flavoring agents, colorants, preservatives stabilizer, thickening agents, chelating agents, buffering agents, tonicity agents, pH adjusting agents, ointment base and vehicle.

2. The formulation of claim 1, wherein the pharmaceutically acceptable salt of 3-hydroxy-2,4,6-trimethylpyridine is selected from the group of salts consisting of succinate, maleate, tartrate, oxalate, fumarate, citrate, hydrochloride, salicylate, pamoate, hydrogen sulfate, sulfate methanesulphonate and benzenesulfonate.

3. The formulation of claim 1, wherein said formulation is in a dosage form selected from the group of dosage forms consisting of tablet, oral liquid, capsule, powder, dry syrup, granules, ointment, gel, cream, ophthalmic preparation, injection, spray, emulsion and suspension.

4. The formulation of claim 1, wherein the diluent is at least one selected from the group consisting of microcrystalline cellulose, starches, lactose, mannitol, calcium phosphate, dibasic calcium phosphate and mixture thereof.

5. The formulation of claim 1, wherein the disintegrant is at least one selected from the group consisting of starches, clays, cellulose derivatives, gums, aligns including alginic acid, combinations of hydrocarbonates with weak acids, crospovidone, sodium starch glycolate, agar, cation exchange resins, citrus pulp, veegum HV, bentonite, cross-linked polyvinylpyrrolidone, carboxymethyl starch, natural starch, microcrystalline cellulose, cellulose gum, carboxymethylcellulose calcium, carboxymethylcellulose sodium, colloidal silica, colloidal silicon dioxide, croscarmellose sodium, guar gum, polacrilin potassium, pregelatinized starch, sodium alginate and sodium starch glycolate.

6. The formulation of claim 1, wherein the lubricant is at least one selected from the group consisting of magnesium stearate, calcium stearate, glyceryl monostearate, glyceryl palmitostearate, stearic acid, talc, zinc stearate, stearic acid, magnesium lauryl sulfate, and colloidal silicon dioxide.

7. The formulation of claim 1, wherein the binder is at least selected from the group consisting of acacia, sodium alginate, starch, gelatin, pregelatinized starch, partly pregelatinized starch, saccharides, glucose, sucrose, dextrose, lactose, molasses, panwar gum, guar gum, ghatti gum, carboxy methylcellulose, methylcellulose, veegur, polyethylene glycols, ethylcellulose, polyvinylpyrrolidone, hydroxypropyl cellulose, hydroxypropyl methylcellulose, starch, gum arabic and dextrin.

8. The formulation of claim 1, wherein the surfactant is at least one selected from the group consisting of alkyl polyethylene oxide, alkylphenol polyethylene oxide, sodium laureth sulphate, sodium dodecyl sulphate, alkyl alcohol, sodium lauryl sulfate, polyoxyethylene block polymers, polyoxypropylene block polymers (poloxamers), glycerols, polyglycerols, fatty acids, polyethylene glycol hydroxystearate, polyalkyl glucosides, ceramides, polyethylene glycol/alkyl glycol copolymers, and polyethylene glycol/polyalkylene glycol ether di-block or tri-block copolymers, diacetylated monoglycerides, diethylene glycol monostearate, ethylene glycol monostearate, glyceryl monooleate, propylene glycol monostearate, macrogol esters, macrogol stearate, polyoxyethylene 50 stearate, macrogol ethers, cetomacrogol 1000, lauromacrogols, nonoxinols, octoxinols, tyloxapol, polyvinyl alcohols, polysorbate, sorbitan monolaurate, sorbitan monooleate, sorbitan monopalmitate, sorbitan sesquioleate, sorbitan trioleate, sorbitan tristearate, sucrose esters, cetyl alcohol, oleyl alcohol, cetylpyridinium chloride, cetyl trimethylammonium bromide, tween 20 and tween 80.

9. The formulation of claim 1, wherein the glidant is at least one selected from the group consisting of colloidal silicon dioxide, magnesium trisilicate, powdered cellulose, starch, talc, tribasic calcium phosphate, lactose, stearates, dibasic calcium phosphate, magnesium carbonate, magnesium oxide, calcium silicate and silicon dioxide aerogels.

10. The formulation of claim 1, wherein the coating polymer is at least one selected from the group consisting of polymethacrylate, polymethamethacrylate, methylcellulose, ethyl cellulose, hydroxymethyl cellulose, hydroxymethylpropylcellulose, cellulose acetate phthalate, arabinogalactan, carboxymethylcellulose, gelatin, gum arabic, polyvinyl alcohol, polyamide, silicones, polyvinyl acetate, hydroxypropyl methylcellulose acetate, rosin, partially hydrogenated rosin and glycerol esters of rosin.

11. The formulation of claim 1, wherein the plasticizer is at least one selected from the group consisting of glycerol, polyethylene glycol, propylene glycol, sugar solution, alcohol, sorbitol, diethyl butyl pthalate, silicone, hexanol, pentanol, dimethylsulfoxide, hexane, oil and mixtures thereof.

12. The formulation of claim 1, wherein the effervescent agent is at least one selected from the group consisting of citric acid, tartaric acid, sodium bicarbonate, potassium bicarbonate and calcium carbonate.

13. The formulation of claim 1, wherein the preservative is at least one selected from the group consisting of benzalkonium chloride, benzyl alcohol, methyl paraben, propyl paraben, butyl paraben, chlorobutanol, metacresol, phenylmercuric nitrate, phenylmercuric acetate, phenylmercuric borate, thiomersal, myristylgamma picolonium chloride, phenol, benzoxonium chloride, cetrimide, phenyl ethanol, chlorohexidine, sorbic acid, potassium sorbate and sodium perborate.

14. The formulation of claim 1, wherein the stabilizer is at least one selected from the group consisting of thiourea, thiosorbitol, sodium dioctyl sulfosuccinate or monothioglycerol, sodium edetate, creatinine, glycine, niacinamide, sodium acetyltryptophanate, sodium caprylate, citric acid and salts thereof.

15. The formulation of claim 1, wherein the thickening agent is at least one selected from the group consisting of ethyl cellulose, methyl cellulose, carbopol, hydroxypropyl methylcellulose, hydroxypropyl cellulose, sodium carboxy methyl cellulose, hydroxyethyl cellulose, polyethylene glycol, acrylates, methacrylates, gelatin, alginates, pectins, tragacanth, karaya gum, xanthan gum, carrageenin, agar, alginate, chitosan, eudrajit and acacia.

16. The formulation of claim 1, wherein the chelating agent is at least one selected from the group consisting of edetate disodium, edetate calcium disodium and edetate tetrasodium.

17. The formulation of claim 1, wherein the buffering agent is at least one selected from the group consisting of acetic acid, adipic acid, benzoic acid, sodium benzoate, citric acid, lactic acid, maleic acid, potassium phosphate, sodium phosphate, sodium acetate, sodium bicarbonate, sodium carbonate, sodium citrate, sodium tartarate, tartaric acid, sodium citrate dehydrate, sodium acetate and sodium acetate trihydrate.

18. The formulation of claim 1, wherein the tonicity agent is at least one selected from the group consisting of glycerin, propylene glycol, polyethylene glycol, lactose, mannitol, dextrose, sodium chloride, potassium chloride, calcium chloride, magnesium chloride, sodium sulfate and sorbitol.

19. The formulation of claim 1, wherein the pH adjusting agent is at least one selected from the group consisting of sodium hydroxide, hydrochloric acid, triethanolamine, ammonia and mixtures thereof.

20. The formulation of claim 1, wherein the ointment base is at least one selected from the group consisting of isopropyl myristate, myristyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, ethylhexyl cocoate, dicaprylyl carbonate, cetearyl isononanoate, oleyl erucate, erucyl oleate, erucyl erucate, octyldodecanol, polydecenes, squalane, dicaprylyl ether, triisostearine, butylene glycol dicaprylate/dicaprate, caprylic/capric triglyceride, olive oil, sunflower oil, soybean oil, peanut oil, rapeseed oil, almond oil, palm oil, coconut oil, palm karnel oil, caster oil, wheat germ oil, grape seed oil, thistle oil, silicon oils, lanolin oil, avocado oil, macadamia oil, candelilla wax, carnauba wax, bees wax, espartograss wax, cork wax, guaruma wax, rice germ oil wax, sugarcane wax, berry wax, ouricury wax, montan wax, jojoba wax, shea butter, beeswax, shellac wax, spermaceti, wool wax, uropygial grease, ceresin, ozocerite, paraffin waxes, sunflower wax, lemon wax, grape fruit wax and laurel wax.


SEQUENCE LISTING (1 sequence)

SEQ ID NO: 1 - protein, 4 aa, Artificial Sequence
  Description of Artificial Sequence synthetic peptide
AEDG